(19) United States
(12) Patent Application Publication
(10) Pub. No.: US 2026/0286079 A1
(43) Pub. Date: Sep. 24, 2026

(54) Direct Laser Processing and Functionalizing Polymer Composites for an On-Demand, Programmable, Recyclable Device Platform

(71) Applicant: The Penn State Research Foundation, University Park, PA (US)

(72) Inventors: Huanyu Cheng, University Park, PA (US); Jia Zhu, Chengdu (CN); Farnaz Lorestani, University Park, PA (US); Yuan Lin, Chengdu (CN)

(73) Assignee: The Penn State Research Foundation, University Park, PA (US)

(21) Appl. No.: 19/575,300

(22) Filed: Mar. 23, 2026

Related U.S. Application Data
(60) Provisional application No. 63/776,457, filed on Mar. 24, 2025.

(51) Int. Cl. C08J 7/12; A61B 5/145; A61B 5/1486; C01B 32/184; C01B 32/194

(52) U.S. Cl. CPC C08J 7/123; A61B 5/14532; A61B 5/1486; C01B 32/184; C01B 32/194; A61B 2562/125; C01P 2006/40; C08J 2379/08; C08J 2383/04; C08J 2479/02

(57) ABSTRACT

Embodiments relate to flexible electronic devices and associated fabrication methods that employ direct laser scribing to create porous laser-induced graphene (LIG) networks on an adhesive, stretchable polymer composite. In particular, the method includes forming a mixture including one or more polymer precursors capable of LIG, one or more elastomeric prepolymers, and one or more adhesion-promoting additives; curing the mixture to form a polymer composite; and laser scribing the polymer composite to pattern an integrated LIG network. The resulting LIG network can be selectively functionalized with nanomaterials, enzymes, or other agents to enable a variety of functions.

### CROSS-REFERENCE TO RELATED APPLICATIONS

This application claims priority to U.S. Provisional Patent Application No. 63/776,457, which was filed on Mar. 24, 2025. The entirety of this application is incorporated by reference herein.

### STATEMENT REGARDING FEDERALLY SPONSORED RESEARCH DEVELOPMENT

This invention was made with government support under Grant Nos. 2319139, DGE2243979, ECCS2222654 and ECCS2309323 awarded by the National Science Foundation and under Grant No. EB030140 awarded by the National Institutes of Health. The Government has certain rights in the invention.

### FIELD

The present disclosure relates to skin-interfaced, flexible electronic devices formed from laser scribing polymer composites. Applications of the device include multifunctional sensing, bioelectronic applications, and other uses.

### BACKGROUND

Flexible electronics capable of directly interfacing with soft and irregular human tissues have garnered significant interest for applications in personal health monitoring and clinical diagnostics. These devices can non-invasively measure various physiological parameters, offering advantages such as conformability, comfort, and real-time data acquisition. The fabrication of functional components, including sensors, electrodes, and interconnects, on pliable substrates typically involves techniques such as photolithography, transfer printing, or direct printing. However, these methods often require complex mask-based processes or specialized nanomaterials with specific viscosity and surface energy properties, which can lead to high manufacturing costs and limited scalability.

As an alternative, mask-free laser fabrication techniques have emerged as promising approaches for the mass production of flexible electronic components due to their high precision, efficiency, and cost-effectiveness. Laser scribing of carbon-rich polymers can induce the formation of porous, conductive three-dimensional graphene networks, known as laser-induced graphene (LIG). These networks offer a large specific surface area suitable for chemical and electrochemical sensing applications. Sensing materials can be deposited onto LIG via dip-coating or chemical reduction, and further modifications, such as doping with metal oxides or decorating with nanomaterials, have demonstrated capabilities in gas sensing, glucose detection, and energy storage devices.

Despite these advances, challenges remain in achieving intrinsically stretchable and conformal bio-sensing interfaces. While LIG can be transferred onto stretchable elastomers, infiltration of elastomer precursors into the porous structure often diminishes porosity, surface area, and hydrophilicity, thereby degrading sensing performance and complicating subsequent doping processes. Direct fabrication of porous, stretchable LIG directly on flexible substrates is thus highly desirable. Existing approaches, such as fabricating LIG on PVA/PI composites for supercapacitors, do not support direct skin contact for biosensing due to water solubility issues. Additionally, integrating multiple functional components, such as sensors, power sources, and interconnects, often requires separate fabrication processes, leading to device complexity, size, and mechanical mismatch at interfaces, which can compromise durability and performance.

Efforts to develop unified material systems, such as nanocomposites with silver nanomaterials or MXene-based systems, have shown promise for strain sensing and energy storage. However, these approaches often involve complex, costly processes and limited tunability. Moreover, the lack of adhesion and compactness in some systems restricts their practical application in skin-interfaced electronics. Consequently, there remains a significant need for low-cost, efficient fabrication methods to produce multifunctional, skin-compatible electronic devices based on a single, intrinsically adhesive material platform.

### SUMMARY

The present disclosure relates to versatile, skin-attachable devices that leverage direct laser patterning and post-processing modifications of polymer composites. These composites are based on laser-induced graphene (LIG) polymer precursors embedded within an elastomer matrix, supplemented with adhesion-promoting additives, such as polyethylenimine ethoxylated (PEIE), to enhance adhesion and functional properties. The laser patterning process creates porous, three-dimensional LIG networks on surfaces of the composites, which can be selectively modified to serve as sensors, electrical interconnects, or other devices.

The intrinsic adhesion properties of the composites, contributed by additives that modulate the crosslinking density of the elastomer, enable direct skin attachment and durable interfacing with human tissue. The porous LIG structures facilitate facile post-processing modifications, such as functionalization with nanomaterials or enzymes, to enable sensitive electrochemical detection of physiological markers, including electrolytes, glucose, and lactate. Additionally, the laser-written patterns can be erased and rewritten through simple tape-assisted removal, providing a recyclable and reconfigurable platform suitable for various multifunctional applications.

This mechanism supports the seamless integration of multiple electronic modules, such as sensors, energy harvesting systems, and wireless communication units, within a compact, flexible, and adherent device. The device's reprogrammability and multifunctionality pave the way for advanced real-time health monitoring, disease diagnostics, and therapeutic interventions.

In an exemplary embodiment, a method of fabricating a device, the method includes forming a mixture including: one or more polymer precursors capable of forming laser-induced graphene (LIG), one or more elastomeric prepolymers, and one or more adhesion-promoting additives; curing the mixture to form a polymer composite; and laser scribing the polymer composite to pattern an integrated LIG network.

In some embodiments, the polymer precursors are selected from the group consisting of polyimide, polyamide-imide, polyether sulfone, polyphenylene sulfide, polyethylene terephthalate, polyethylene naphthalate, and combinations thereof.

In some embodiments, the polymer precursors include polyimide.

In some embodiments, the elastomeric prepolymers are selected from the group consisting of polydimethylsiloxane, thermoplastic polyurethane, styrene-ethylene-butylene-styrene, and combinations thereof.

In some embodiments, the elastomeric prepolymers include polydimethylsiloxane.

In some embodiments, the adhesion-promoting additives are selected from the group consisting of polyethylenimine ethoxylated, polyethyleneimine, polydopamine, tannic acid, and combinations thereof.

In some embodiments, the adhesion-promoting additives include polyethylenimine ethoxylated.

In some embodiments, the polymer precursors include polyimide, the elastomeric prepolymers include polydimethylsiloxane, and the adhesion-promoting additives include polyethylenimine ethoxylated.

In some embodiments, the mixture includes from 50 wt % to 75 wt % of the polymer precursors and from 25 wt % to 50 wt % of the elastomeric prepolymers, relative to the total combined weight of the polymer precursors and elastomeric prepolymers.

In some embodiments, the mixture includes 25 μL to 30 μL of the adhesion-promoting additives per 10 g of the elastomeric prepolymers.

In some embodiments, the method further includes functionalizing the LIG network with conductive nanomaterials.

In some embodiments, the conductive nanomaterials are selected from the group consisting of carbon nanotubes, graphene oxide, and reduced graphene oxide.

In some embodiments, the conductive nanomaterials are metal nanoparticles selected from the group consisting of gold, silver, platinum, palladium, manganese, zinc, copper, nickel, cobalt, iron, titanium, molybdenum, tungsten, magnesium, aluminum, lead, and combinations thereof.

In some embodiments, the method further includes functionalizing the LIG network with enzymes.

In some embodiments, the enzymes are selected from the group consisting of glucose oxidase and lactate oxidase.

In some embodiments, the method further includes peeling off and removing an uppermost portion of the polymer composite with the integrated LIG network; and laser scribing an underlying portion of the polymer composite to pattern a second integrated LIG network.

In an exemplary embodiment, a device is fabricated from the method described above.

In some embodiments, the device exhibits intrinsic adhesion and stretchability.

Further features, aspects, objects, advantages, and possible applications of the present disclosure will become apparent from a study of the exemplary embodiments and examples described below, in combination with the Figures, and the appended claims.

### BRIEF DESCRIPTION OF THE DRAWINGS

The above and other objects, aspects, features, advantages, and possible applications of the present innovation will be more apparent from the following more particular description thereof, presented in conjunction with the following drawings. Like reference numbers used in the drawings may identify like components.

FIG. 1 is a schematic illustration showing an exemplary fabrication process of a polymer composite and device.

FIG. 2 is a flow chart demonstrating an exemplary fabrication process of a polymer composite and device.

FIG. 3 is a radar chart of various LIG forms, including those derived from a polymer inventive composite, a PI film (LIG@PI), and transferred to PDMS (LIG@PDMS).

FIG. 4 is an optical image of a PDM/PEIE-based device patch for biopotential regulation and measurement. The bottom panel shows the multifunction device patch being attached to skins, under deformations induced by poking, and during peeling off.

FIG. 5 is a schematic illustration showing a multifunctional adhesive device patch with biopotential measurement, electrical stimulation, biosensing, thermal management, and wound treatment capabilities.

FIG. 6 is a graph showing the peeling-off force of PI_0.5/PDMS/PEI_30 normalized by the width in the adhesion measurement.

FIG. 7 is a graph showing the adhesive strength of a PI/PDMS composite with varying amounts of PEIE additives.

FIG. 8 is a graph showing stress-strain curves of the PI_0.5/PDMS composites with varying amounts of PEI additives.

FIG. 9 is a graph showing the modulus of a PI/PDMS composite with varying amounts of PEIE additives.

FIG. 10 includes graphs showing (left) modulus and (right) adhesion strength of the PI/PDMS/PEI_25 composite with varying PI fractions: 0, 0.25, 0.5, 0.75 (mass ratio to PDMS), compared with pure PDMS.

FIG. 11 shows optical images of the PI/PDMS/PEIE composite with different PI fractions (i.e., 20, 50, and 75%).

FIG. 12 includes (left) an optical image and (right) the water permeability of the porous PI/PDMS/PEIE compared with that of PI/PDMS/PEIE and PDMS.

FIG. 13 shows scanning electron microscopy (SEM) images of LIG fabricated from the adhesive PI/PDMS/PEIE composite.

FIG. 14 shows the Raman spectrum of LIG fabricated from the adhesive PI/PDMS/PEIE composite.

FIG. 15 demonstrates the influence of (left) image density and (right) laser power on the conductive property or sheet resistance of the LIG. It is noted that the low image density (i.e., 3, 4, and 5) leads to disconnected LIG, whereas the high image density of 7 gives overburnt patterns.

FIG. 16 is a graph showing the measured sheet resistance of LIG fabricated from the PI/PDMS/PEIE composite with different PI fractions and laser scribing directions (i.e., longitudinal or transverse).

FIG. 17 is a graph showing the electromechanical response of LIG fabricated from the PI_0.5/PDMS composite with longitudinal or transverse laser scribing.

FIG. 18 includes graphs showing durability tests of the LIG@PI/PDMS/PEIE under 1,000 cycles of stretching and releasing (10%), with the zoom-in views of the first and last 10 cycles in the left and right panels.

FIG. 19 shows the hydrophilicity of LIG fabricated from the PI_0.5/PDMS/PEIE composite with different PI fractions and the contact angle of LIG fabricated from the PI_0.5/PDMS/PEIE composite as a function of time.

FIG. 20 shows the contact angle of the transferred LIG on PDMS (i.e., LIG@PDMS).

FIG. 21 shows the contact angle of pristine PDMS after laser scribing.

FIG. 22 shows the contact angle measurements of LIG fabricated from PI_0.5/PDMS with (top) or without (bottom) PEI (25 L per 10 g PDMS) over 2 hours (i.e., 0, 20, 40, 60, 80, 100, and 120 mins).

FIG. 23 shows the contact angle measurements of LIG fabricated from PI_0.5/PDMS with PEI over days (i.e., 1, 2, 3, 4, and 5 days).

FIG. 24 shows optical images of the device patch for ECG or EMG measurements upon various deformations.

FIG. 25 is a graph showing a comparison of ECG signals captured by the commercial electrode and the patch during the first and second use, and after reprinting. Artificial deformations were introduced to compare the robustness of various LIG-based bioelectrodes during ECG measurements. The ECG signals under stretching and compression deformations were magnified in the left and right panels, respectively.

FIG. 26 shows (left) optical images and (right) ECG signals measured by the LIG@PI (i.e., LIG directly fabricated from PI films) and LIG@PDMS (i.e., LIG transferred to PDMS) electrodes before, during, and after deformations.

FIG. 27 shows (top) an experimental setup of ECG measurements by the LIG@PI/PDMS/PEIE-based adhesive patch with an integrated flexible, miniaturized, wireless data processing unit and built-in Bluetooth module. Also shown are the measured (bottom left) ECG and (bottom right) extracted heart rate (HR) from the ECG of a subject before and after a workout.

FIG. 28 shows EMG measurements by the LIG@PI/PDMS/PEIE-based adhesive patch in both dry and wet environments.

FIG. 29 is a graph showing EMG signals measured by the LIG@PI/PDMS/PEI-based adhesive composite during the short and long clench.

FIG. 30 shows optical images of the skin with the LIG@PI/PDMS/PEI-based adhesive composite applied for 0, 12, and 24 hours, showing no sign of irritation.

FIG. 31 shows a demonstration of the recycled PI/PDMS/PEI-based composite by an erasing and rewriting process.

FIG. 32 includes graphs showing biopotential measurements by a recycled PI/PDMS/PEI-based composite. (left) ECG signals measured by the reprinted composite (1^{st}, 2^{nd} and 3^{rd} times) before, during, and after deformations. (right) EMG signals measured by the composite in both dry and wet environments.

FIG. 33 is a graph showing a comparison of the CV curves between LIG@PDMS and LIG@PI/PDMS/PEI in a Prussian blue deposition solution.

FIG. 34 is a schematic of the electrochemical sensing mechanism.

FIG. 35 is a graph showing the open-circuit potential (OCP) of the PANI/AuNP/LIG-based electrode versus an Ag/AgCl quasi-reference electrode.

FIG. 36 includes graphs showing (left) a CV curve of the GOx/PB/AuNP/LIG electrode in 0.2M PBS (pH 6.5) in the presence of 1 mM glucose with a scan rate of 50 mV/s; (middle) the amperometry response of the working electrode to consecutive injection of glucose into the stirred 0.2M PBS (pH 6.5) with an applied potential of 0.26 V (i.e., the oxidation peak) and (right) the data fitted to the Michaelis-Menten equation by using non-linear regression analysis for GOx and the corresponding calibration curve.

FIG. 37 includes graphs showing (left) a CV curve of the LOx/PB/AuNP/LIG electrode in 0.2M PBS (pH 6.5) in the presence of 1 mM lactate with scan rate 50 mV/s; (middle) Amperometry response of the working electrode to consecutive injection of lactate into the stirred 0.2M PBS (pH 6.5) with an applied potential of −0.1 V (i.e., the reduction peak) and (right) the corresponding calibration curve.

FIG. 38 shows optical images of the LIG electrode modified by electroless plating of nickel (top), air-spraying of silver nanowires (AgNWs) (middle), and drop-casting of Ag nanoparticle (AgNP) ink (bottom).

FIG. 39 includes a graph and images showing the heating performance of the Ni/LIG-based flexible heater.

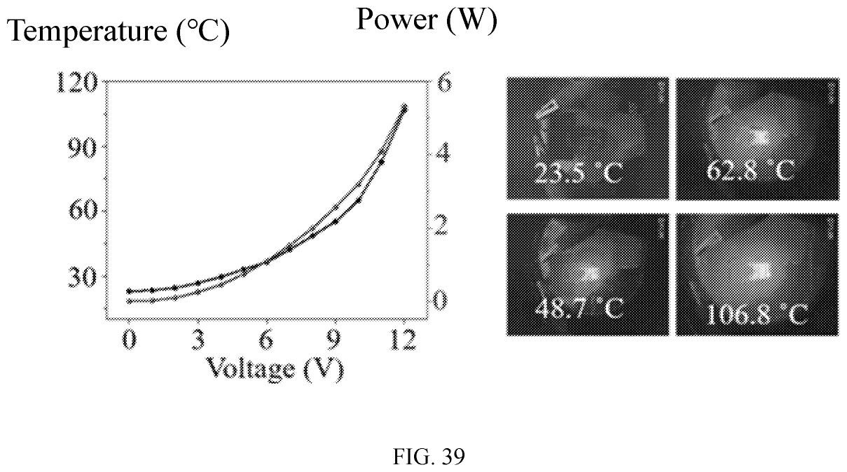

FIG. 40 is a graph showing S₁₁ curves of a wideband AgNW/LIG-based dipole antenna with double-arm configurations under bending deformations.

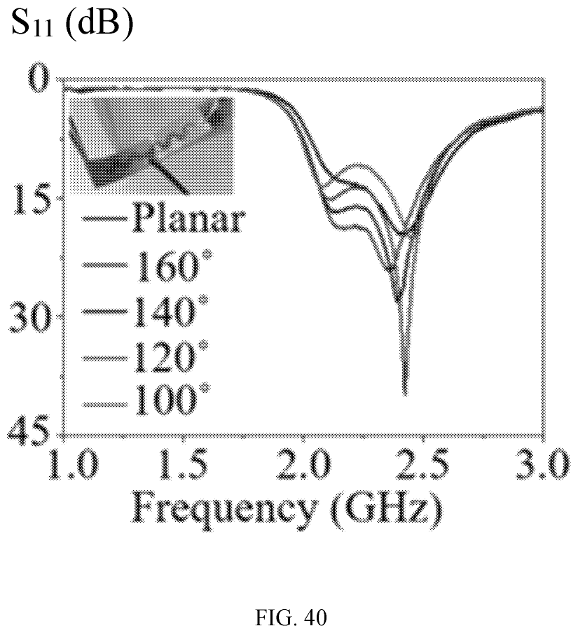

FIG. 41 is a graph showing the resonant frequency of an AgNW/LIG-based NFC device under bending deformations determined by the “Min-phase” method.

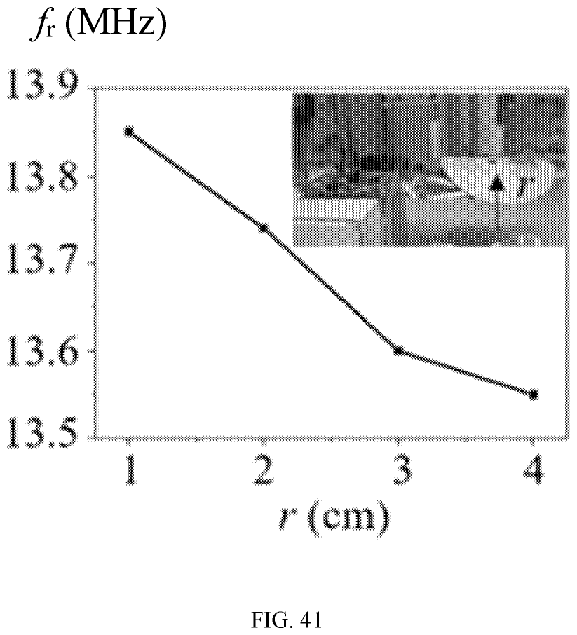

FIG. 42 shows optical images of the NFC device wirelessly powered by a transmitter to light an LED.

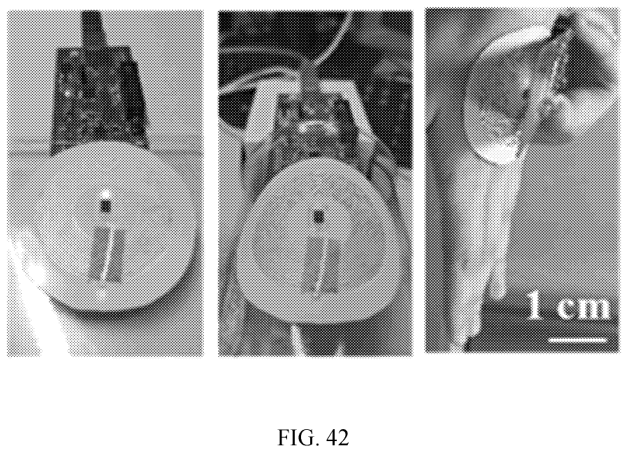

FIG. 43 shows SEM images of LIG modified by nickel (Ni), silver nanowires (AgNWs), and silver nanoparticles (AgNPs) at low (top) and high (bottom) magnifications.

FIG. 44 shows simulated (top left) S₁₁, (top right) Zn, and (bottom) 3D radiation patterns of the wideband dipole antenna based on AgNP/LIG.

FIG. 45 is a graph showing measured S₁₁ curves of five antennas.

FIG. 46 is a graph showing the measured impedance of the AgNP/LIG-based NFC coil. The dashed line indicates the resonant frequency of the NFC coil.

FIG. 47 is a graph showing phase curves of the AgNW-LIG-based NFC coil fabricated on PI/PDMS/PEI before and after bending with bending radii of 4, 3, 2, and 1 cm.

FIG. 48 shows optical images of the fluorescein release upon an applied voltage of −1.5 V.

FIG. 49 shows a schematic of the drug delivery device and the corresponding circuit model.

FIG. 50 shows optical images of the dual-functional LIG@PI/PDMS/PEIE-based patch with wirelessly controlled drug delivery and UV-LED indicator.

FIG. 51 is a graph showing the output voltage (Vout) as a function of the distance between the device and transmitter.

FIG. 52 is a graph showing the antibacterial performance of the dual-functional LIG@PI/PDMS/PEIE patch.

FIG. 53 shows a skin-interfacing real-time sweat glucose sensing patch based on LIG@PI/PDMS/PEIE. (left) Optical images of the sweat glucose sensing patch and experimental setup for real-time measurements. (right) Real-time sweat (C_{sweat}) and blood (C_{blood}) glucose concentration during indoor workouts measured by the patch and commercial finger-prick blood test, respectively.

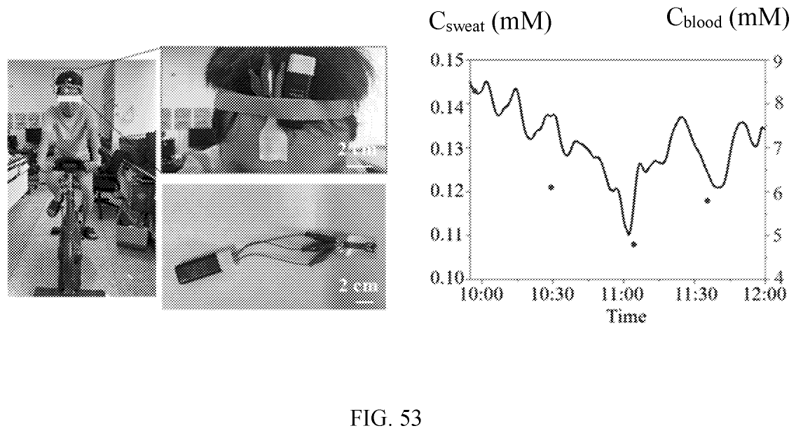

FIG. 54 shows optical images of (left) PI/thermoplastic polyurethane (TPU) and (right) PI/styrene-ethylene-butylene-styrene (SEBS) composites.

FIG. 55 is a graph showing the power spectral density (PSD) of ECG and EMG signals measured by the LIG@PI/PDMS/PEIE-based adhesive patch.

### DETAILED DESCRIPTION

The following description illustrates exemplary embodiments and methods of use that are presently contemplated for implementing the present invention. This description is not intended to be limiting, but rather to elucidate the general principles and features of various aspects of the invention. The scope of the invention is not restricted by this description.

Embodiments relate to flexible electronic devices and to associated fabrication methods. Methods employ direct laser scribing to create porous, conductive laser-induced graphene (LIG) networks on an adhesive, stretchable polymer composite. The resulting LIG patterns can be selectively functionalized with nanomaterials, enzymes, or other agents to enable a variety of functions. The polymer composites and laser processing steps enable fabrication of an intrinsically adhesive, conformal, recyclable, and programmable device platform suitable for direct skin attachment and long-term wear.

Unless otherwise specified, all technical and scientific terms used herein have the meanings commonly understood by persons skilled in the art to which this invention pertains. Any definitions provided herein are intended to supplement these common understandings and are not meant to be limiting.

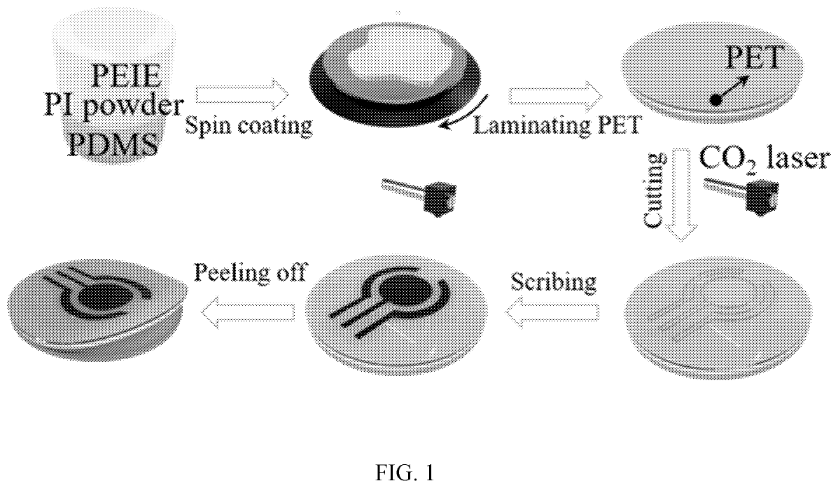

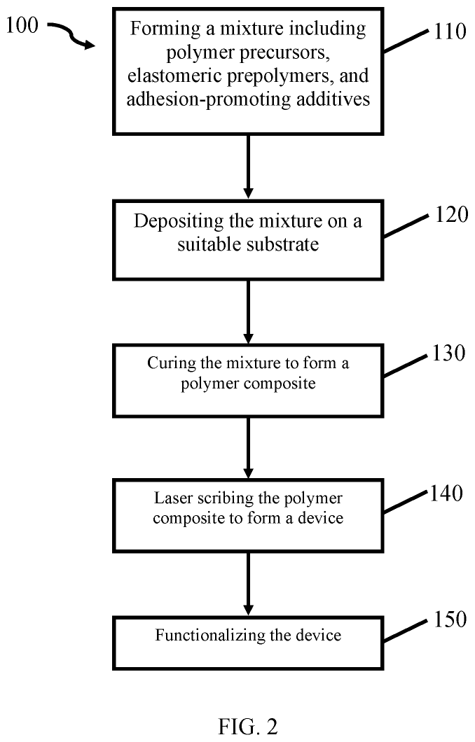

Referring to FIGS. 1 and 2, an exemplary method 100 of forming a flexible electronic device includes forming a mixture 110. The mixture includes one or more polymer precursors, one or more elastomeric prepolymers, and one or more adhesion-promoting additives.

The polymer precursors serve as the feedstock for forming porous graphene structures (i.e., LIG) upon laser scribing. The precursors may include, but are not limited to, polyimide (PI), polyamide-imide (PAI), polyether sulfone (PES), polyphenylene sulfide (PPS), polyethylene terephthalate (PET), polyethylene naphthalate (PEN), or combinations thereof.

The elastomeric prepolymers provide flexibility and stretchability to the device. Examples of suitable prepolymer include, but are not limited to, silicone elastomers (e.g., polydimethylsiloxane (PDMS), Ecoflex®, Dragon Skin®, RTV silicones, fluorosilicones, modified silicones, among others), thermoplastic polyurethane (TPU), styrene-ethylene-butylene-styrene block copolymer (SEBS), styrene-butadiene-styrene block copolymer (SBS), thermoplastic polyolefins (TPO), thermoplastic vulcanizates (TPV), and polyester elastomers, acrylic and methacrylate elastomers including soft acrylics, PSA-type elastomers, and UV-curable systems; rubber-based elastomers such as natural rubber, nitrile rubber (NBR), butyl rubber, EPDM, neoprene, and hydrogenated rubbers; hydrogel or ionic elastomers including physically or chemically crosslinked hydrogels; as well as composite, filled, porous, gradient-modulus, self-adhesive, self-healing, multilayered, or surface-modified elastomers, or other elastomeric materials capable of forming stretchable matrices.

The adhesion-promoting additives can modulate both adhesion and mechanical properties of the polymer composite. In particular, they may enhance interfacial adhesion through chemical interactions with the elastomer matrix and modify the crosslinking density, thereby tuning adhesion strength and mechanical compliance. Suitable additives may include, but are not limited to, polyethylenimine ethoxylated (PEIE), poly(ethylene glycol) (PEG), polyethyleneimine (PEI), polydopamine (PDA), tannic acid, or other suitable additives capable of promoting adhesion.

The mixture may include from 50 wt % to 75 wt % of the polymer precursors and from 25 wt % to 50 wt % of the elastomeric prepolymers, relative to the total combined weight of the polymer precursors and elastomeric prepolymers.

In nonlimiting embodiments, the mixture may include no less than 50 wt %, no less than 55 wt %, no less than 60 wt %, no less than 65 wt %, no less than 70 wt %, and/or the like, of the polymer precursors, relative to the total combined weight of the polymer precursors and elastomeric prepolymers. In further nonlimiting embodiments, the mixture may include no greater than 75 wt %, no greater than 70 wt %, no greater than 65 wt %, no greater than 60 wt %, no greater than 55 wt %, and/or the like, of the polymer precursors, relative to the total combined weight of the polymer precursors and elastomeric prepolymers.

In nonlimiting embodiments, the mixture may include no less than 25 wt %, no less than 30 wt %, no less than 35 wt %, no less than 40 wt %, no less than 45 wt %, and/or the like, of the elastomeric prepolymers, relative to the total combined weight of the polymer precursors and elastomeric prepolymers. In further nonlimiting embodiments, the mixture may include no greater than 50 wt %, no greater than 45 wt %, no greater than 40 wt %, no greater than 35 wt %, no greater than 30 wt %, and/or the like, of the elastomeric prepolymers, relative to the total combined weight of the polymer precursors and elastomeric prepolymers.

The mixture may include 25 μL to 30 μL of the adhesion-promoting additives per 10 g of the elastomeric prepolymers.

In nonlimiting embodiments, the mixture may include no less than 25 μL, no less than 26 μL, no less than 27 μL, no less than 28 μL, no less than 29 μL, and/or the like, of the adhesion-promoting additives per 10 g of the elastomeric prepolymers. In further nonlimiting embodiments, the mixture may include no greater than 30 μL, no greater than 29 μL, no greater than 28 μL, no greater than 27 μL, no greater than 26 μL, and/or the like, of the adhesion-promoting additives per 10 g of the elastomeric prepolymers.

The method 100 further includes depositing the mixture onto a suitable substrate 120. Any appropriate deposition technique may be used, such as spin coating, to form a uniform, thin film.

The mixture is then cured to form a polymer composite 130. The composite includes the polymer precursors and the adhesion-promoting additives dispersed within an elastomeric matrix derived from the elastomer prepolymers.

Next, laser scribing is performed on the polymer composite to pattern a device with an integrated LIG network 140. During laser scribing, the polymer precursors undergo liquefaction and carbonization, resulting in a porous, multilayered LIG network with high surface area and electrical conductivity. The laser parameters, including power, scan speed, and pattern design, can be optimized to conductive patterns with desired dimensions, porosity, and/or electrical properties.

After laser patterning, the polymer composite can be peeled from the substrate, yielding a flexible, stretchable, and highly adhesive device.

In certain embodiments, the LIG structure can be “erased” by peeling off with an adhesive tape, and subsequently re-patterned via laser scribing. This reusability supports device reprogramming or recycling, with the composite maintaining its mechanical and electrical performance over multiple cycles with minimal degradation.

The device's flexibility and stretchability enable conformal attachment to skin or other irregular surfaces. The adhesion-promoting additives enhance interfacial adhesion, allowing the device to adhere strongly to curved and moving surfaces while maintaining low impedance and high signal fidelity during motion. The composite's low elastic modulus and high stretchability also support long-term wear without delamination or discomfort.

The laser-scribed LIG network can further be functionalized with various materials 150. Its hierarchical, porous, interconnected 3D structure enhances hydrophilicity and provides active sites for subsequent functionalization. The functionalization materials that may be functionalized on the LIG are not particularly limited and may depend on the intended application of the device. The materials may include, but are not limited to, conductive nanomaterials, enzymes, or biorecognition elements.

In some embodiments, the LIG network is modified with conductive nanomaterials, such as carbon nanotubes (CNTs), graphene oxide (GO), or reduced graphene oxide (rGO). Metal nanoparticles, including gold, silver, platinum, palladium, manganese, zinc, copper, nickel, cobalt, iron, titanium, molybdenum, tungsten, magnesium, aluminum, and/or lead, can also or alternatively be incorporated to enhance electrochemical properties. These nanoparticles may take the form of nanoshells, nanorods, nanorings, or core-shell structures, and other particulate forms.

In some embodiments, the LIG network is modified with enzymes, such as glucose oxidase (GOx), lactate oxidase (LOx), or other biorecognition elements. Such embodiments may enable the device to be used for specific biomarker or biochemical detection. For example, lactate oxidase can be immobilized on the surface of the device to catalyze the oxidation of lactate, producing hydrogen peroxide as a byproduct. The device can then detect this hydrogen peroxide electrochemically.

As described above, the device described herein exhibits a broad spectrum of potential applications across biomedical, clinical, personal health, and technological domains.

Sensing Elements and Sensors: Devices can be configured as sensors capable of detecting a wide range of physiological signals. For example, the porous LIG network provides a high surface area with excellent electrical conductivity and hydrophilicity, which can be exploited to create electrophysiological sensors for monitoring bioelectrical signals such as electrocardiograms (ECG), electromyograms (EMG), electrooculograms (EOG), and electroencephalograms (EEG). The composite's intrinsic adhesion properties enable secure, comfortable, and durable attachment of the sensors directly onto the skin, ensuring stable electrical contact. The sensors enable continuous, real-time recording of electrical activity during daily activities or clinical procedures, facilitating diagnostics, health monitoring, and disease management.

In addition to electrophysiological signals, devices can serve as biochemical sensors for detecting analytes in bodily fluids such as sweat, saliva, etc. As previously explained, functionalization with enzymes like glucose oxidase (GOx), lactate oxidase (LOx), or other biorecognition elements allows for specific detection of biomarkers such as glucose, lactate, pH, sodium, potassium, chloride, and other electrolytes or metabolites.

Bioelectronic and Clinical Applications: Devices can be employed in various bioelectronic applications, including the delivery of electrical stimulation for neuromodulation or muscle activation, as well as for wound healing. Due to the device's conformal adhesion to skin or tissue, combined with its biocompatibility, the devices can be applied directly to wounds, ulcers, or surgical sites. Devices can also provide real-time monitoring of wound parameters such as pH, temperature, and infection biomarkers. Furthermore, devices can facilitate electrically controlled drug delivery, such as localized administration of antibiotics, growth factors, or anti-inflammatory agents, by incorporating wireless energy transfer and controlled release mechanisms.

Energy Harvesting and Self-Powered Systems: A device's porous LIG network can be functionalized to serve as a flexible, lightweight energy harvester, converting biomechanical energy into electrical power. Integrating triboelectric nanogenerators (TENGs), piezoelectric materials, or other energy harvesting components within the composite can enable the device to generate power from natural body movements, such as walking, joint motion, or respiration. This harvested energy can power the sensors, wireless modules, or therapeutic components, creating a self-sustaining, autonomous system capable of continuous operation without external power sources.

Wireless Communication and Data Transmission: A device's porous LIG network can be doped or functionalized to act as RF antennas, NFC coils, or low-loss transmission lines, facilitating wireless data transmission and energy transfer. This enables real-time, remote monitoring of physiological and biochemical data, which can be wirelessly relayed to external devices such as smartphones, tablets, or cloud-based servers. Such capabilities support telemedicine, remote diagnostics, and personalized health management, allowing users and clinicians to access vital information conveniently and promptly.

### EXAMPLES

The following examples provide exemplary embodiments of the present invention, and the examples are not intended to limit the scope of the invention in any way.

### Experimental Section

Fabrication of LIG@, PI/PDMS/PEIE: The fabrication of elastomeric bio adhesives started with manual mixing of PDMS (Sylgard 184), PI powder (Evonik, P84 NT, 6 m), and PEIE with varying mass ratios. A mass mixing ratio of 1:0.25, 1:0.5, and 1:0.75 of PDMS to PI powder was studied. The PEIE was added to the mixture to reduce the crosslinking of PDMS for improved adhesion of the resulting composite. The PDMS/PI/PEIE mixture diluted by heptane was spin-coated on a silicon wafer treated with a sacrificial PVA layer. After spin coating for 60 s at a speed of 500 rpm, the wafer was transferred to an oven at 80° C. for 1 hour. Increasing the curing time resulted in decreased adhesion and increased stiffness. A thin PET film of 20 μm was attached to the cured composite to avoid potential contamination and maintain the adhesion of the composite in the following processes. A CO₂ laser with a wavelength of 10.6 um and a spot size of ~130 μm was used to first cut the PET film in the vector mode to expose the underlying composite and then create LIG patterns in the raster mode (power of 10.5%, speed of 11%, PPI of 1000, and image density of 6). Peeling the resulting film off the silicon wafer created porous LIG in PDMS/PEIE. The remaining PET would serve as a mask in the following steps. Gentle air flow was employed to remove the LIG debris before chemical modifications or measurements.

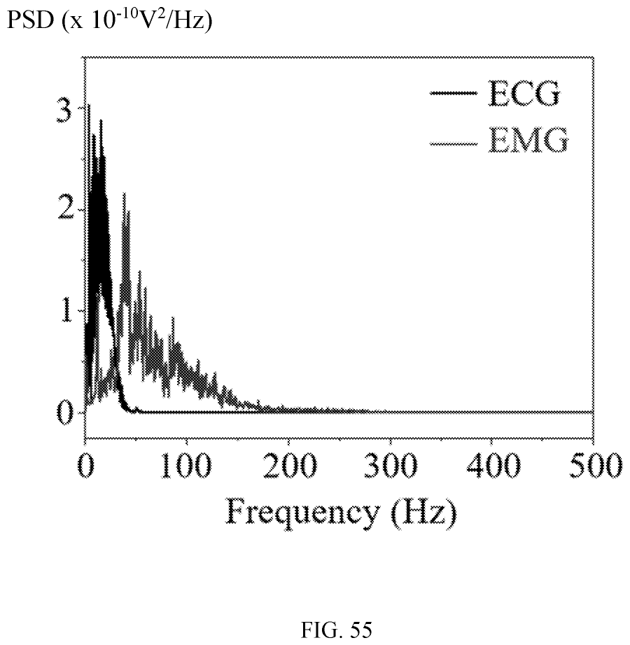

Biopotential measurements with the LIG@PI/PDMS/PEIE: After wiring with silver pastes, the LIG@PI/PDMS/PEIE adhesive patch was applied to clean skin treated with alcohol wipes. A multi-channel data acquisition system (PowerLab 16/35) combined with an amplifier (Bio Amp) was used to measure ECG and EMG at a sampling rate of 1000 Hz. An analog/hardware bandpass filter (10-200 Hz) implemented in Bio Amp was applied before the A/D conversion for the raw ECG and EMG sampling. The small baseline drift in the raw ECG/EMG data indicated effective suppression of both the thermal noise and Flicker noise. The SNR of raw EMG signals was calculated as the ratio of the root-mean-square value of the raw EMG signal during muscle contraction to that at rest. The power spectral density of raw ECG and EMG signals was obtained by using Welch's method implemented in MATLAB with a fast Fourier transform (FFT) size of 1000 and 50% window overlap (FIG. 55). The high-pass digital filters were not applied to remove Flicker noise from the raw data due to their strong coupling with the signal in the low-frequency range. The SNR of ECG signals was calculated as the ratio of the power in the frequency range of interest (0-100 Hz) to that in the high-frequency range (i.e., the thermal noise). The SNR induced by the motion artifacts was calculated by following the method reported previously. In brief, the noise induced by the motion artifacts was first obtained by applying a low-pass filter (1 Hz). The ECG and EMG signals were obtained by subtracting the noise from the raw data. The SNR corresponding to motion artifacts was calculated as the ratio of the root-mean-square value of the signal after subtracting the noise to that of the noise.

Fabrication of LIG-based REs: A drop of AgNP ink was added to the as-fabricated composite electrode, followed by annealing in an oven at 80° C. for 10 min. Next, the electrode was immersed in a FeCl₃ solution for 1 min for colorization and then rinsed with DI water. After drying in air, 20 μl PVB cocktail solution (79.1 mg of PVB, 50 mg of NaCl, 1 mg of F127, and 0.2 mg of multiwalled CNT in 1 ml of methanol) was drop-cast on the electrode. Afterward, 10 μl diluted Nafion solution was coated on the PVB-modified electrode to complete the fabrication of LIG-based RE.

Fabrication of LIG-based WEs for pH and Glucose/Lactate Sensing: For pH sensing, the obtained electrode was then modified by electroplating PANI. After mixing 0.1 M aniline and 1 M H₂SO₄ to prepare the electroplating solution, CV deposition of PANI from −0.2 to 1 V with respect to a commercial Ag/AgCl reference electrode was performed at a scan rate of 0.05 V/s for 30 cycles, followed by rinsing with DI water. The characterization of PANI was carried out by CV in 1 M HCl with a scan rate of 0.1 V/s. pH measurements were carried out by connecting WE and RE in a two-electrode system.

The fabrication of the WE was conducted under the in-situ electrodeposition of PB and AuNPs. The electrodeposition was performed by cyclic voltammetry (CV) between −0.2 and 0.8 V (versus Ag/AgCl) at a scan rate of 0.1 V/s⁻¹ for 90 cycles in a freshly prepared mixed solution of 250 mg/L HAuCl₄, 2×10⁻³ M FeCl₃, 2×10⁻³ M K₃[Fe(CN)₆], 0.1 M KCl, 0.01 M HCl, and DI water. After electrodeposition, the as-fabricated WE was rinsed with DI water and dried in ambient conditions. Then, the PB/AuNP/LIG electrode was modified by GOx (concentration of 34 mg/ml in DI water) mixed with the chitosan solution at a 1:1 wt. % ratio. The chitosan solution was prepared by mixing 2 wt. % chitosan and 2 wt. % acetic acid solution with an additional 0.8 wt. % glycerol in DI water at 80° C. Similarly, mixing the obtained chitosan solution with LOx (34 mg/mL in DI water) at a 1:1 wt. % ratio prepared the LOx solution. The completed LOx solution was stored at −10° C. for future use after complete mixing. After drop-casting the prepared LOx solution on the PB/AuNP/LIG WE and drying in the ambient condition, 5 wt. % glutaraldehyde and 1 wt. % Nafion was drop-casted. The resulting WE for lactate sensing was stored at 4° C. overnight before testing.

Fabrication of LIG Composite-based Wiring and Interconnects: Metallic nanomaterials (AgNWs, Ni, and AgNPs ink) were prepared on the LIG composite (with PET as a self-mask to define the patterns) to improve its conductivity for highly conductive electric interconnects or RF applications. Diluted AgNWs (AgNWs-60, ACS material) solution by isopropyl alcohol (IPA) with a ratio of 1/10 (v/v) was sprayed on the LIG composite by a commercial airbrush (G222; Master Airbrush), with the nozzle fixed at a distance of 20 cm from the substrate and an airbrush pressure of ~20 psi. The PET self-mask was removed after the evaporation of IPA. The Ni layer was deposited on the LIG composite through electroless plating, using commercial electroless plating precursor solutions. In brief, after surface activation of the LIG composite by pre-treating with A, B, and C solutions in sequence, immersing in D solution at 65° C. for 30 s completed the electroless plating process. AgNP inks (JS-A911, Novacentix) were manually air-sprayed on the LIG composite. After the solvent evaporation, annealing with a xenon light through photo sintering (Xenon Corporation, X-1100) for three pulses (pulse duration of 529 s, energy of 1000 J, and voltage of 3000 V) yielded a conductive Ag coating on the LIG composite.

Materials Characterization: The Raman spectrum was measured by Horiba (HORIBA, Ltd.) with a 532 nm laser source. SEM characterization of LIG was carried out with TESCAN MIRA3 (TESCAN ORSAY HOLDING). Mechanical stiffness and adhesion measurements were carried out with a universal testing machine (M230Pro, Hengyi Precision Instruments Limited Company), with the stretching and peeling-off rate set to 10 mm/min and 200 mm/min, respectively. The electromechanical properties of the LIG@PI/PDMS/PIEI were carried out by stepwise stretching with a custom-built stretching system and a source meter (Keysight 2902). Fatigue tests were performed by cyclic stretching at a rate of 0.5%/s.

Modification of LIG Electrodes for Drug Delivery: Polypyrrole (PPy) modifications on LIG electrodes for drug delivery were achieved by electropolymerization of pyrrole monomers in a three-electrode configuration with a commercial Ag/AgCl reference electrode and a Pt counter electrode. A constant current of 0.1 mA/cm² was applied to LIG in a solution containing 0.1 M pyrrole and 0.01 M cefazolin sodium for 360 s. Next, cefazolin antibiotics were loaded as dopants in PPy by electrodeposition at a constant current density of 0.5 mA/cm² in a solution for 360 s with 0.1 M pyrrole and 0.01 M cefazolin sodium. Afterward, another layer of PPy was electropolymerized as encapsulations with the same setup. The electrode was then rinsed with DI water. The PPy/fluorescein layer was electrodeposited with the same procedure except that 0.01 M cefazolin sodium was replaced by 0.1 M fluorescein. The obtained working electrode was combined with a bare LIG counter and reference electrodes in a three-electrode configuration.

Antibacterial Tests: Four devices (i.e., Group I, II, III, and IV) were first treated by alcohol and UV sterilization before being attached to the bottom of Petri dishes for bacterial culture. Agar was uniformly applied on the device for grafting of bacterial suspension, followed by bacterial culture in the incubator. A wireless transmitter was placed under the petri dish in Groups I, II, and III to trigger the LED illumination and/or drug release for 1 hour. After a 24-hour culture, the antimicrobial activity was evaluated by measuring the area of the ZOI.

On-body Tests: Before the application of the adhesive LIG@PI/PDMS/PEIE patch for sweat glucose sensing, the forehead skin of the subject was cleaned with alcohol wipes. Then, the adhesive patch was wired with a miniaturized, wireless electrochemical station (BioSYS-P15E Max, Shenzhen Refresh Biosensing Technology Co., Ltd). Afterward, the subject was required to do indoor cycling for sweat sampling and glucose measurements. All human subject studies were approved by the Institutional Review Board at Penn State University.

### Results and Discussion

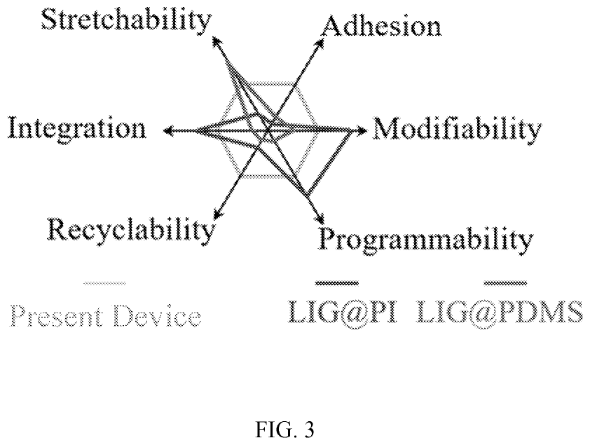

Design Strategy of LIG@, PI/PDMS/PEIE: The stretchable bioadhesive composite is designed by mixing PI powders (size of ~6 m) with PDMS precursors and PEIE additive followed by curing at 80° C. for 3 hours (FIG. 1). The PI microparticles and PDMS serve as the functional component and supporting matrix, respectively, whereas the PEIE additives provide enhanced adhesion by forming a complex with Pt catalyst in PDMS to reduce its crosslinking density. A similar enhanced adhesive strength of PDMS can be achieved by adding polyethylene glycol (PEG). Upon laser scribing, PI microparticles are carbonized to form conductive porous 3D LIG patterns through a liquefaction process arising from a photothermal reaction, whereas PDMS is converted into semiconducting silicon carbide or silica, depending on the environment. Different from the LIG created from the PI film (LIG@PI), the LIG fabricated from the adhesive composite (LIG@PI/PDMS/PEIE) exhibits good stretchability and strong adhesion to directly form conformal contact with human skin. Although stretchability and adhesion can be slightly improved after transferring LIG@PI to PDMS (LIG@PDMS) through an infiltration and separation process, the PDMS on the LIG network significantly reduces the porous structures and post-processability (e.g., coating or functionalization). As a result, LIG@PI/PDMS/PEIE combines and exceeds the figure-of-merits of both LIG@PI and LIG@PDMS to open up additional application opportunities in bioelectronics (FIG. 3 and Table 1). With on-demand laser scribing and facile post functionalization, the adhesive multifunctional skin-interfaced device platform can be fabricated to measure biopotential and other physiologically relevant chemical signals, provide electrical stimulations and drug delivery, and integrate wireless

| | | Adhe- | modi- | pro- | re- | inte- |
|---|---|---|---|---|---|---|
| | Stretch- | sion | fia- | gram- | cycla- | gra- |
| Materials | ability | (N/m) | bility | mability | bility | tion |
| LIG@PI/ | 13% | ~19.8 | Yes | Yes | Yes | Yes |
| PDMS/ | | | | | | |
| PEI | | | | | | |
| LIG@PI | ~1-2% | ~0 | Yes | Yes | No | Yes |
| LIG@ | ~50% | ~0.3 | No | No | No | No |
| PDMS | | | | | | |

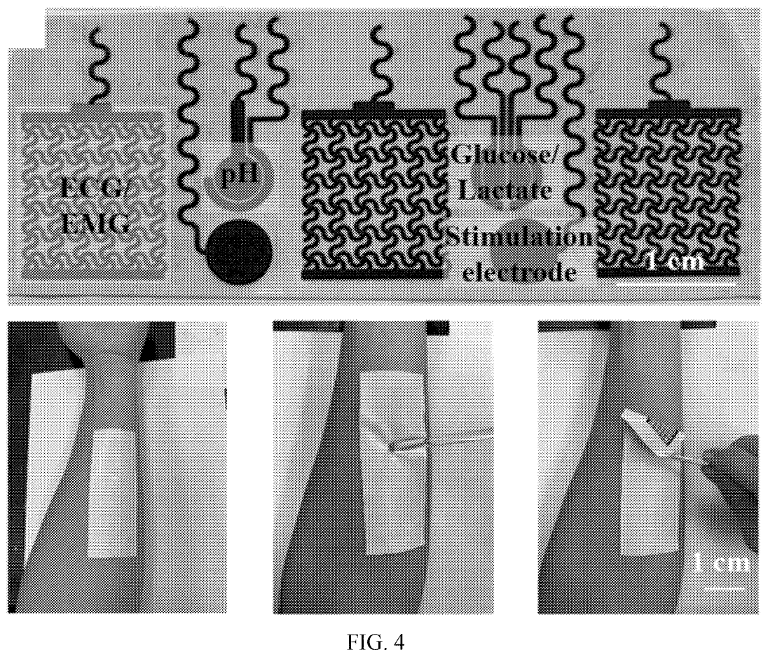

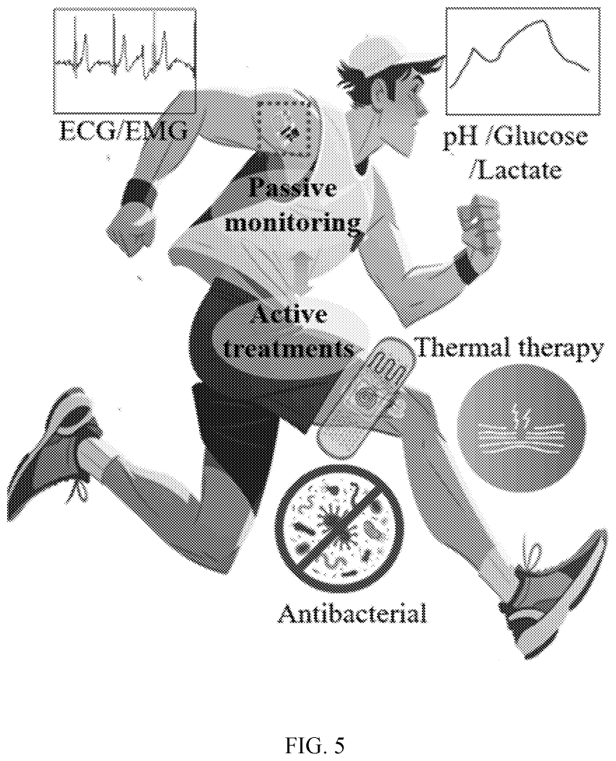

modules for future data/energy transmission (FIG. 4). The post functionalization of the LIG@PI/PDMS/PEIE composite with specific enzymes (or aptamers) provide the highly sensitive and selective electrochemical sensors for glucose and lactate sensing. The highly conductive nanomaterials decorated on the composite allow for motion-artifact-free electrophysiological signal monitoring, efficient joule heating toward thermal therapy, and low-loss RF circuits for wireless communication (FIG. 5). Additionally, simple erasing with peeling off and rewriting with the same laser setup showcase a unique yet efficient fabrication platform to create recyclable and reusable flexible sensors and devices with incomparable programmability at low cost.

Table 1: Comparison between LIG@PI/PDMS/PEIE, LIG@PI, and LIG@PDMS.

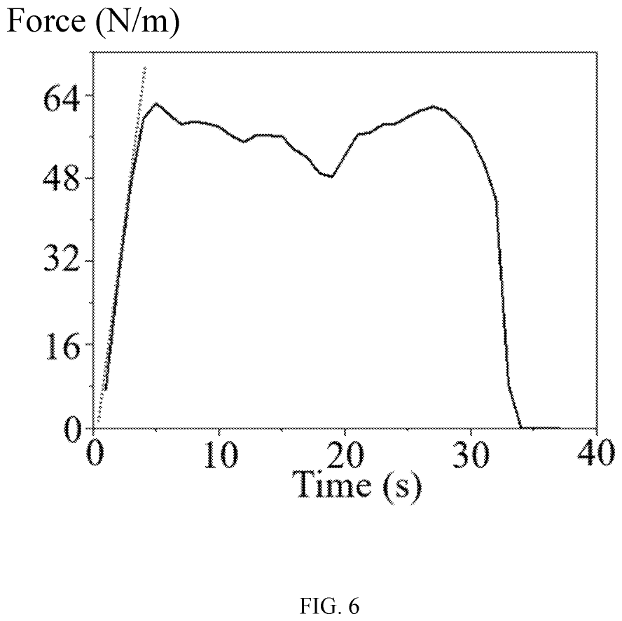

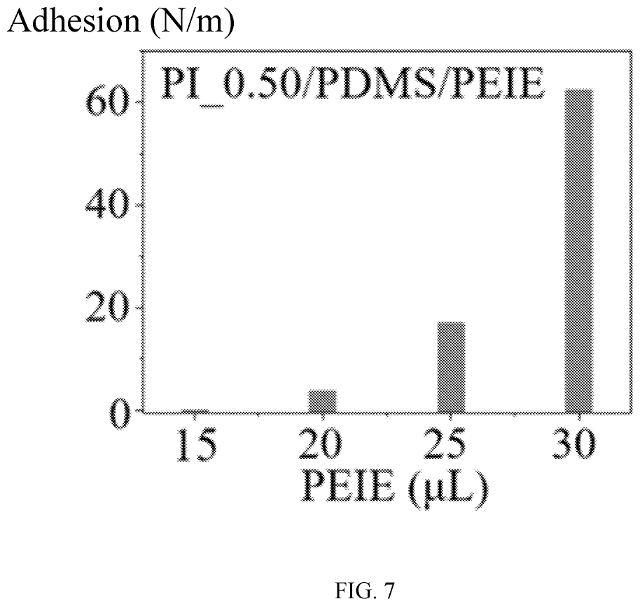

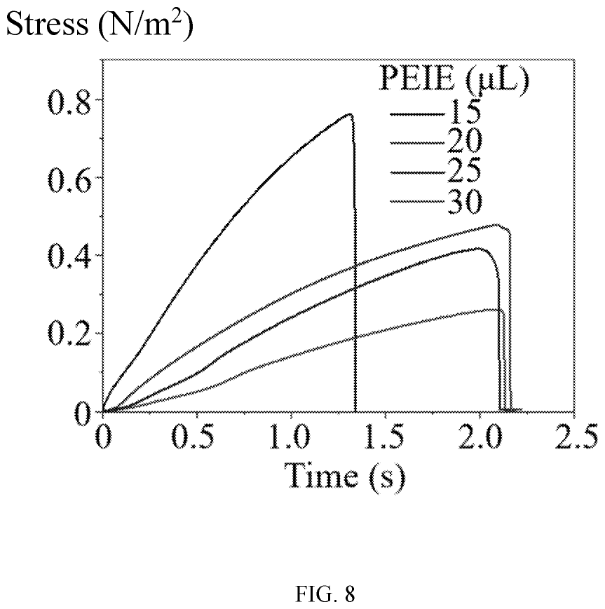

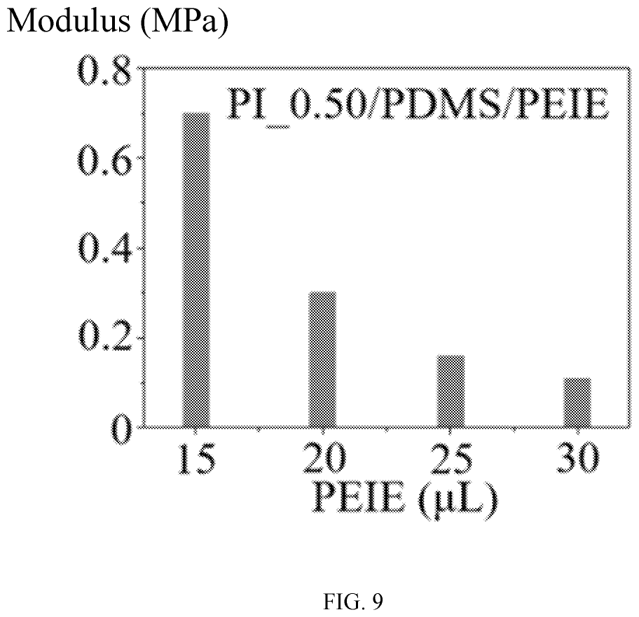

Optimization of Mechanical Stiffness, Adhesion, and Electrical properties of LIG@PI/PDMS/PEIE: PEIE can greatly reduce the crosslinking density of PDMS for enhanced adhesion. As a result, the strong yet tunable adhesive strength of the PI/PDMS/PEIE composite to target surfaces can be easily modulated by changing the PEIE content. With a customized experimental setup, the adhesion of the PI/PDMS/PEIE composite to a glass slide before LIG preparation is experimentally measured during the peeling-off process with the adhesive strength extracted from the plateau of the normalized force per unit width versus time curve (FIG. 6). For the PI/PDMS composite with a mixing weight ratio of 0.5:1 (i.e., PI_{0.5}/PDMS), the adhesion increases significantly from 0.164 to 63.377 N/m as the PEIE content increases from 15 to 30 μL per 10 g PDMS (FIG. 7). It should be noted that the adhesion of PI/PDMS/PEIE deceases after laser scribing as the created LIG in LIG/PI/PDMS/PEIE is non-adhesive. At the same time, Young's modulus (calculated from the slope of the strain-stress curve in FIG. 8) of the resulting composite also decreases from 0.70 to 0.11 MPa due to the incorporation of PEIE (FIG. 9). However, the reduced modulus may not provide sufficient cohesion to maintain the integrity of LIG patterns during peeling-off. As a result, it is important to co-optimize Young's modulus and adhesion of the PI/PDMS/PEIE composite.

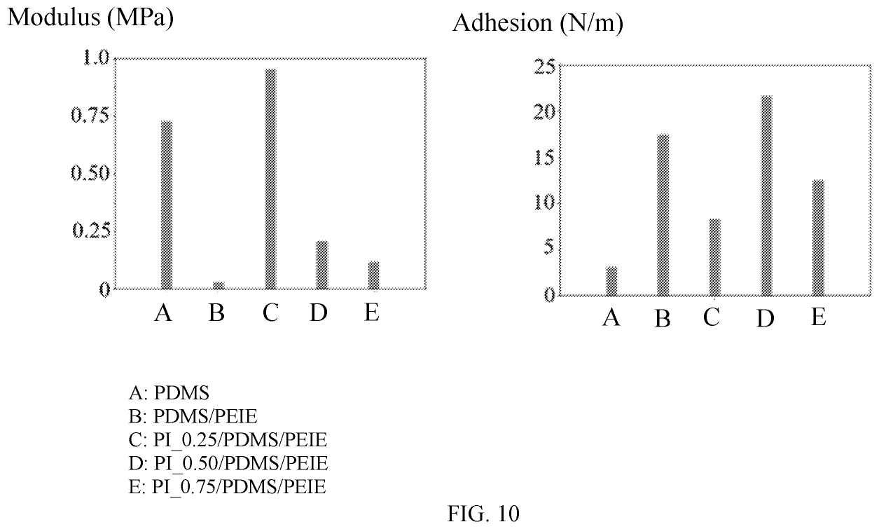

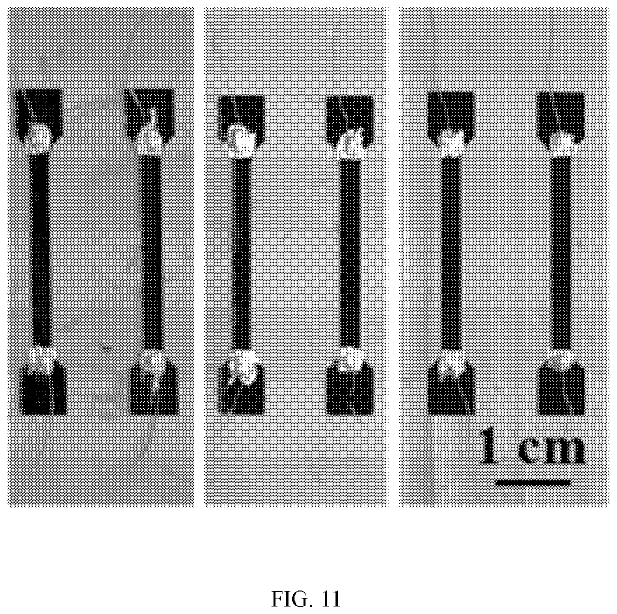

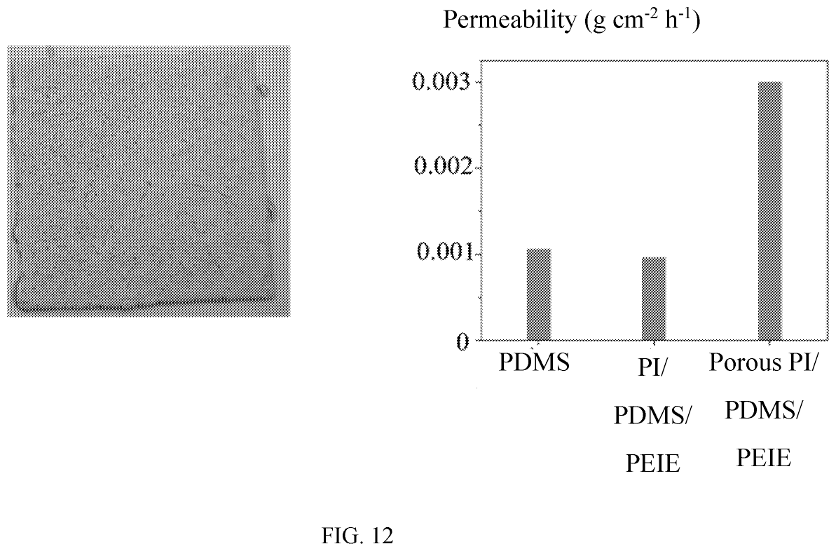

The inherently non-stretchable and non-adhesive PI powders in the composite also modulate the mechanical properties of the composite by providing mechanical reinforcement and reducing the PDMS network density. The modulus of the adhesive PDMS/PEIE without PI powders is less than 0.1 MPa, which is significantly lower than the pristine PDMS (FIG. 10). With 25% PI powders (to PDMS), the modulus is increased to 0.95 MPa, which is even higher than the pristine PDMS, attributing to the reinforcing effect of stiff PI microparticles. However, further increasing PI content to 50% and 75% leads to a slightly reduced modulus of 0.21 and 0.11 MPa, which is inconsistent with the trend reported in previous work. Since there are no direct interactions between PI microparticles, reduced modulus with overloading PI microparticles arises from the low PDMS density in the composite. Compared with PDMS without PI powders (PDMS/PEIE), only the one with 50% PI powders (PI_0.50/PDMS/PEIE) shows a slight improvement of 24% in the adhesive strength (FIG. 10). As a result, the content of both the PI and PEIE can be leveraged to tune the adhesive strength and modulus of the resulting composite. Additionally, the optical reflection of the composite diminishes with the increasing PI content, which results in distinct visual appearances (FIG. 11). Using the same constituent materials and fabrication process can also prepare elastomeric substrates with spatial-dependent mechanical properties and LIG patterns with tunable electrical properties. Meanwhile, highly permeable LIG@PI/PDMS/PEIE can be prepared by adding volatile ethanol into the precursor to introduce porous structures (FIG. 12). The porous structure leads to a three-fold increase in water permeability (~0.003 g cm⁻² h⁻¹) that is comparable to textile-based electronics (~0.004 g cm⁻² h⁻¹), allowing applications in skin-interfaced electronics.

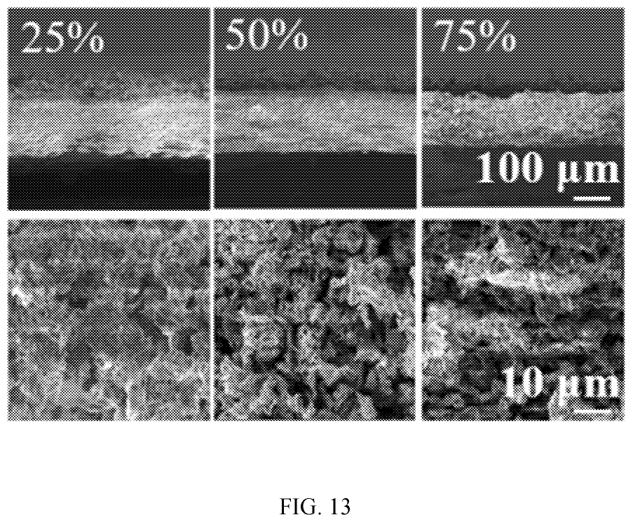

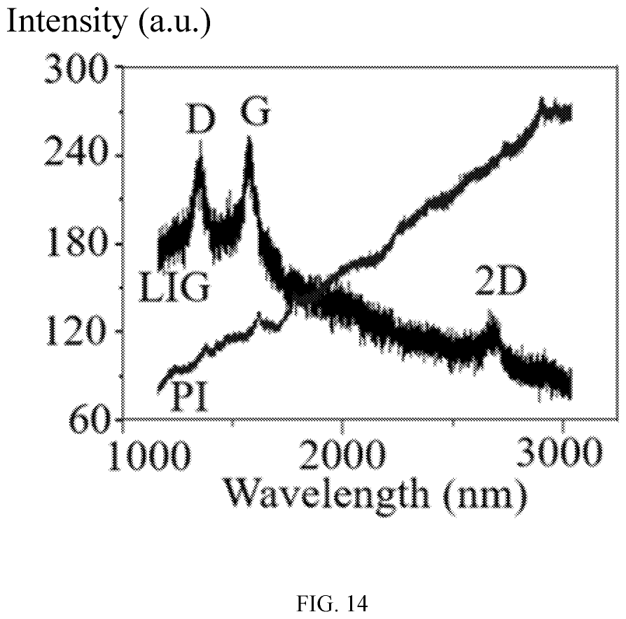

The pristine PDMS is mainly converted to microstructured silica during laser scribing in an ambient environment. The use of PI powders in the composite changes coarse and discontinuous flakes induced by laser scribing to continuous and porous LIG networks, as revealed by the SEM images (FIG. 13). The porosity of the resulting LIG increases with the increasing PI powders, culminating in an average pore size of ca. 10 μm for 75% PI powders, which is much larger than that of LIG@PI. This is due to the lower PI fraction in the PI/PDMS composite than the PI film and the partial block by the non-converted PDMS. Different from the pristine PI/PDMS substrate, the as-fabricated LIG shows three characteristic peaks of graphene at 1356 (D), 1580 (G), and 2700 (2D) cm⁻¹ in the Raman spectrum (FIG. 14), which confirms the formation of few-layered LIG as in the previous study. The ratio of the 2D to G peak of ca. 0.3 indicates the existence of multilayered graphene. The disorders or defects of porous graphene revealed by the D peak provide active sites for sensing. These results confirm the successful conversion from PI particles in the composite to multilayered porous graphene by laser scribing.

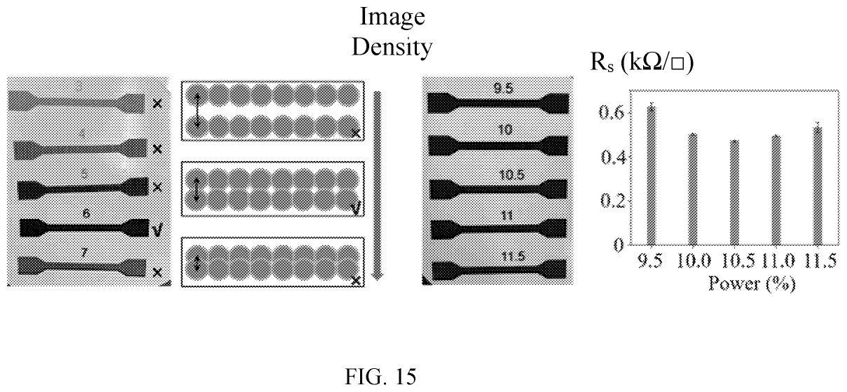

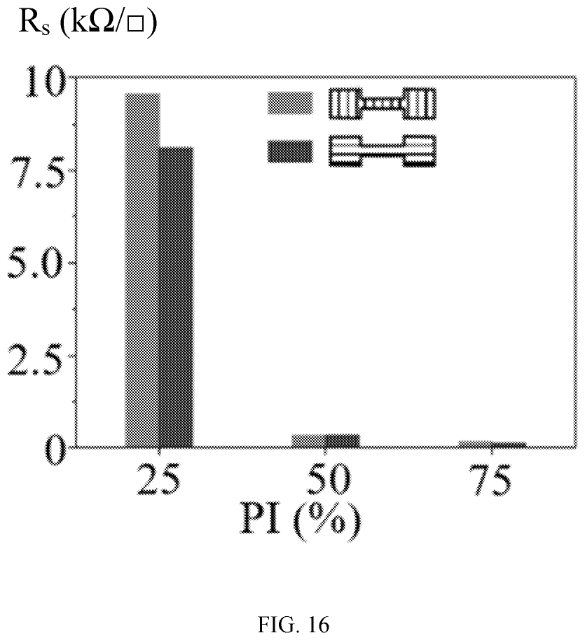

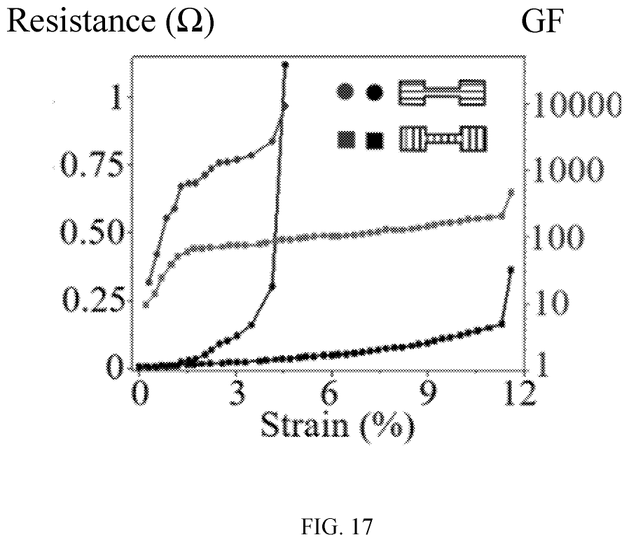

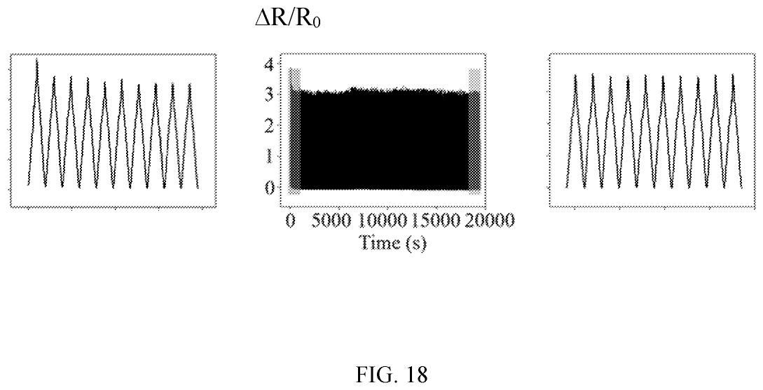

The morphology and electrical properties of the resulting LIG fabricated from the PI/PDMS/PEIE composite, primarily determined by the PI content, are revealed by the electrical and electromechanical characterizations of dog bone-shaped patterns. The laser parameters with an image density of 6 (i.e., 1000 lines per inch) and laser power of 10.5% are optimized to result in improved conductivity of the LIG (FIG. 15). LIG patterns with longitudinal laser scribing have lower sheet resistance than the transversely laser-scribed ones. For example, the sheet resistance (0.175 kΩ/□) of the longitudinally scribed LIG is lower than that (0.22 kΩ/□) of the transversely scribed LIG in the composite with 75% PI powders created at the same laser power and speed. With the same laser scribing parameters, the sheet resistance of the as-fabricated longitudinal LIG decreases from 7.5 to 0.175 kΩ/□ as the PI powders increase from 25% to 75% (FIG. 16). However, the resistance of LIG@PI/PDMS/PEIE shows less dependence on laser scribing directions compared to that of LIG@PI. The as-fabricated LIG@PI/PDMS/PEIE with transverse laser scribing shows a much smaller response to stretching up to 12% than the one with longitudinal scribing for the PI_{0.5}/PDMS sample (FIG. 17), which is consistent with previous reports. Cyclic tests over 1,000 times also reveal the stable electromechanical properties of the LIG@PI/PDMS/PEIE (FIG. 18). The overall stretchability of LIG@PI/PDMS/PEIE can be further increased when combined with stretchable designs such as serpentine, wavy, or kirigami structures. It is worth noting that 12% is sufficient for most on-body applications except for locations with extreme deformations, such as joints. The minimum linewidth obtained by CO₂ laser scribing is around 150 μm, but a higher resolution can be obtained by green, UV, or femtosecond lasers.

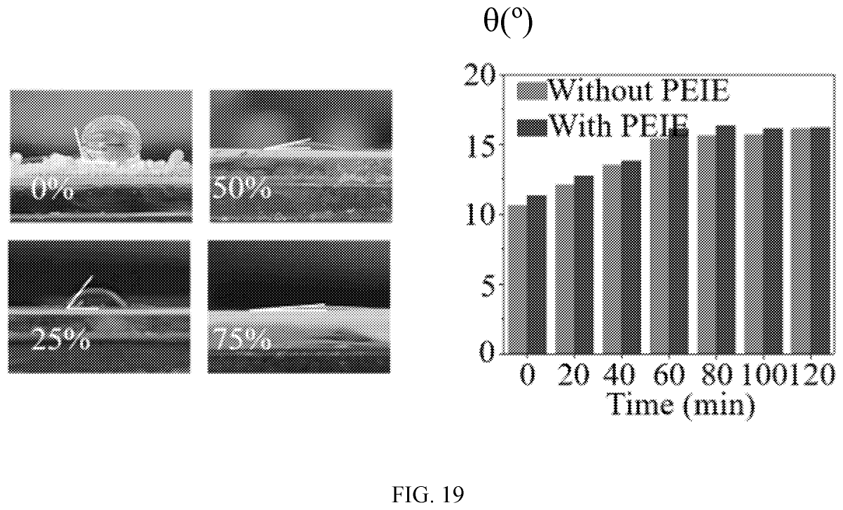

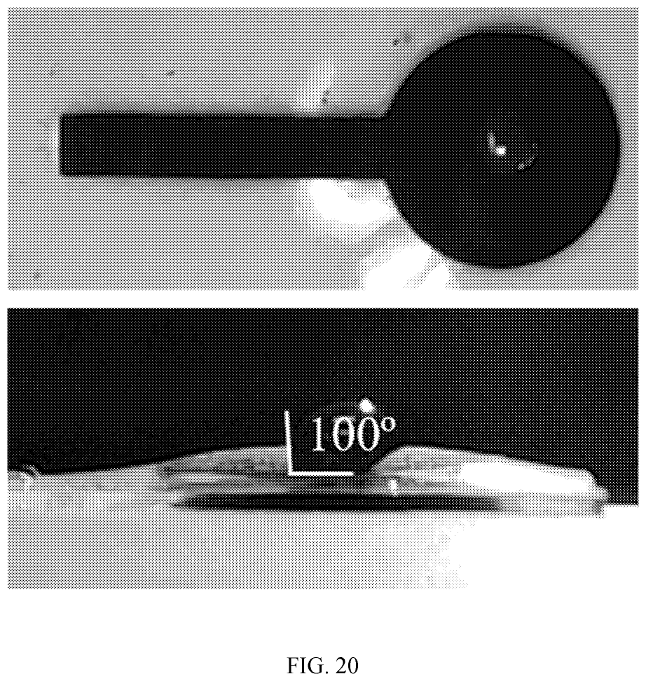

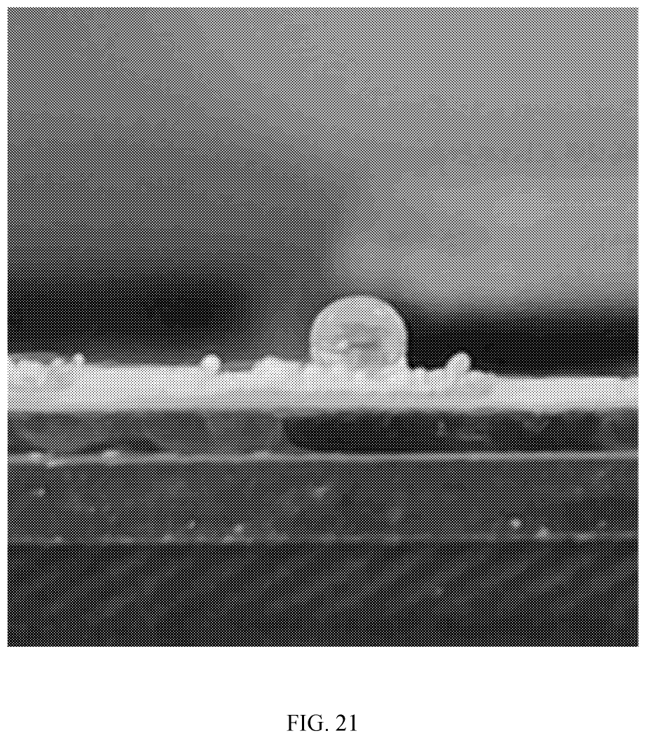

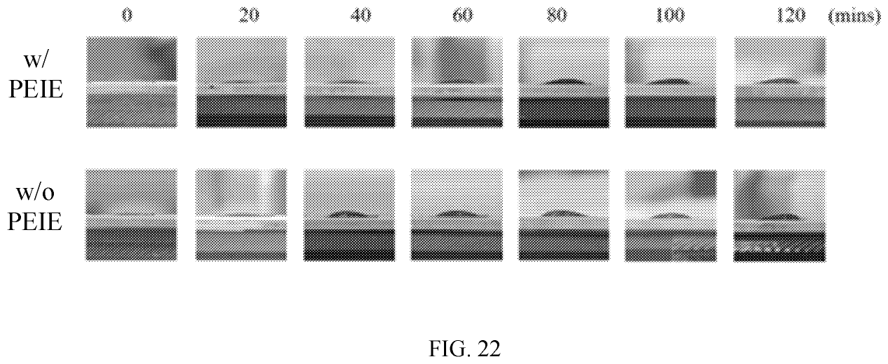

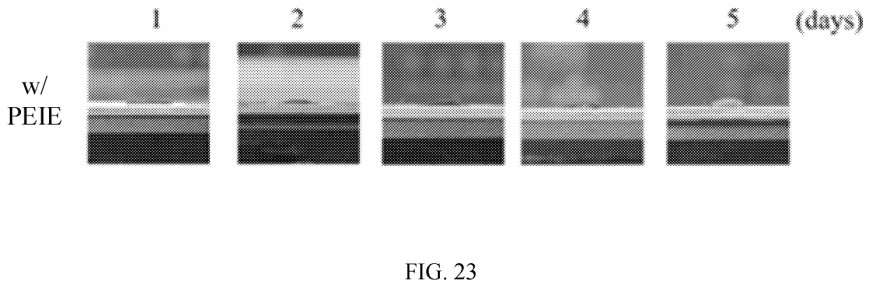

Good hydrophilicity is highly desired for post-functionalization through dip-coating, in situ reduction, or electroplating to prepare LIG@PI/PDMS/PEIE for various biosensing applications. Before laser scribing, the PI/PDMS/PEIE composite with varying PI contents shows a similar contact angle to the pristine PDMS. The conversion of hydrophobic PDMS to SiC by laser scribing results in a slightly increased contact angle from 109° to 113°, indicating degraded hydrophilicity. However, LIG@PI/PDMS/PEIE provides dramatically improved hydrophilicity, with the contact angle decreased from 510 to 6° as the PI content increases from 25 to 75% (FIG. 19). The large difference in surface wettability between LIG and PI/PDMS/PEIE allows the water drop placed at the interface to rapidly and efficiently transported to the LIG side. In comparison, LIG@PDMS has a much larger contact angle of ~100° due to the infiltration of hydrophobic PDMS into the porous LIG (FIG. 20). Meanwhile, PDMS remains highly hydrophobic after laser scribing (FIG. 21), indicating that the improved hydrophilicity of PI/PDMS/PEIE after laser scribing arises from the newly formed LIG. The high hydrophilicity of the LIG@PI/PDM composite with or without PEIE additives can also be maintained during 2-hour measurements (FIGS. 19 and 22) or even over 5 days (FIG. 23). Overall, increasing the PEIE content can greatly improve the adhesive strength and reduce the modulus of the composite. A high PI content leads to high-quality LIG (i.e., good electrical conductivity and porosity), but at the loss of the composite's mechanical stiffness. As the composite with 75% PI or more corresponds to a low mechanical stiffness, the composite with 50% PI is chosen for high LIG quality and mechanical stiffness in the following investigation unless specified otherwise. The incorporation of PEIE increases the adhesion but at a cost of reduced modulus. As a result, 25 μL PEIE per 10 g PDMS is adopted in the following study.

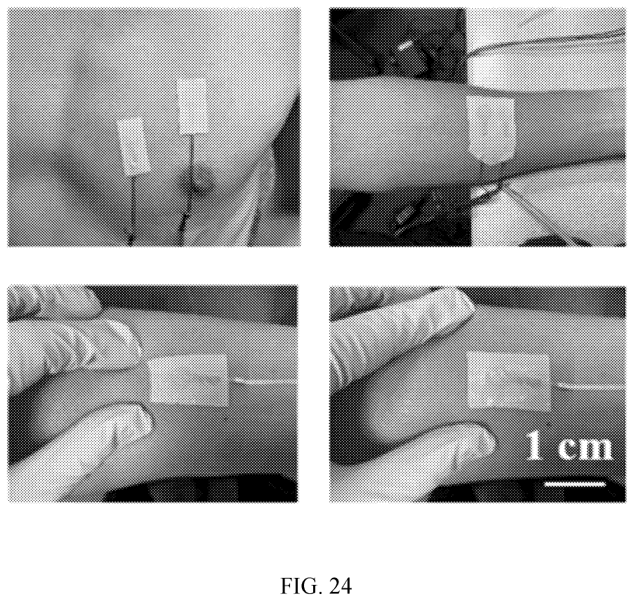

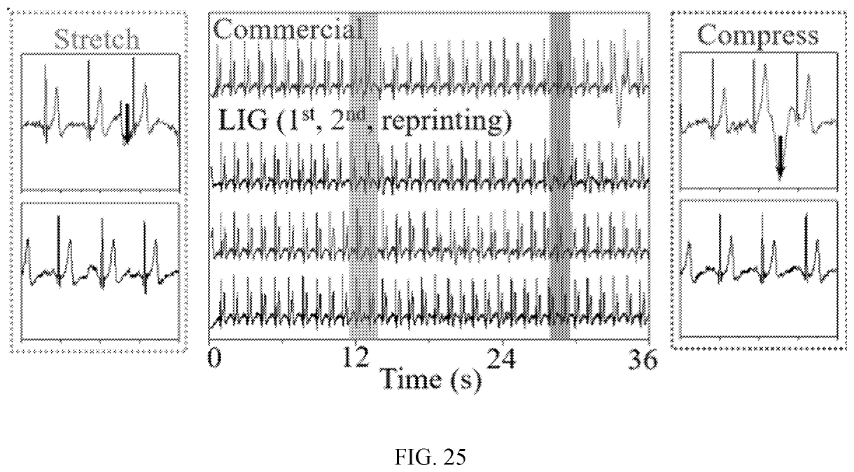

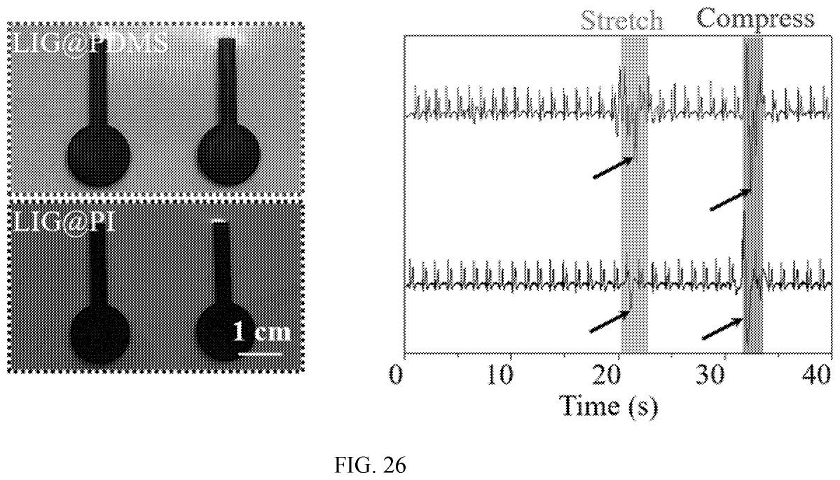

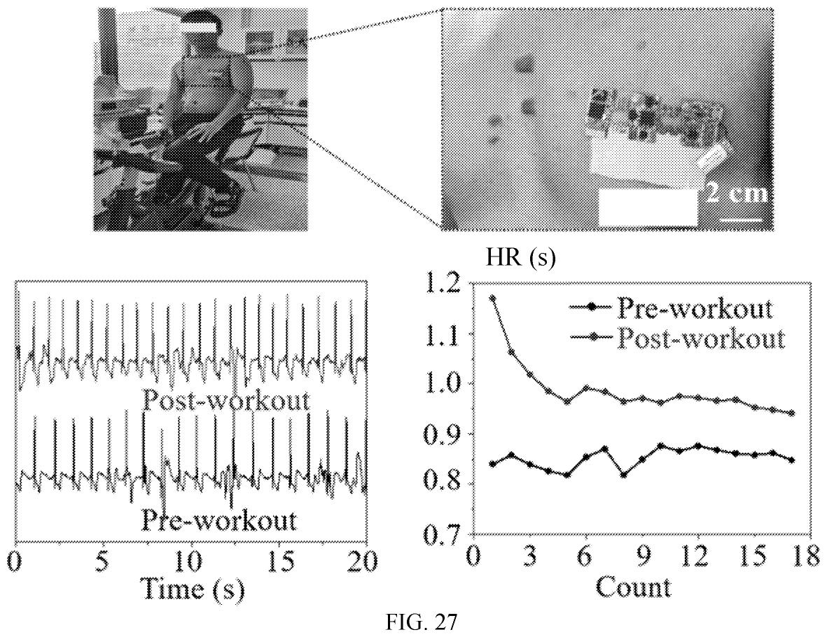

Biopotential Monitoring: The strong adhesion and small bending stiffness of the LIG@PI/PDMS/PEIE patch with a thickness of 100 μm allow facile conformal attachment to curvy human skin or tissues without the need for an additional adhesive layer. It is also worth noting that the PET film also serves as a protection layer to maintain strong adhesion before use on the skin. The adhesive patch strongly conforms to the human chest and arm, even upon daily movements or severe mechanical deformations such as stretching or squeezing (see FIG. 24) due to the good stretchability of the composite. The signal-to-noise ratio (SNR) of 24.7 from the measured ECG signals captured by the stretchable adhesive patch is comparable to that (26.4) of gel electrodes (FIG. 25). The excellent adhesive strength of the patch can last for over 24 hours. Even after one and two cycles of peeling off and reattaching to the human skin, the adhesive patch was capable of maintaining a high SNR of 23.6 and 23.3 in ECG measurement. More importantly, high-fidelity electrophysiological signals such as ECG and EMG can be captured in continuous measurements involving artificial deformations. The electrophysiological signal recorded by the LIG@PI/PDMS/PEIE electrodes during the stretching or compression motions (induced with fingers) shows no abnormal deviations in the sinus electrocardiogram waveform, maintaining a high SNR of 22.8 in a 15-second measurement. In contrast, the commercial dry gel electrodes show signal fluctuations during the manually applied skin deformations (black arrow in the magnified insets) with a low SNR of 15.5. In comparison, electrodes based on LIG@PI or LIG@PDMS (FIG. 26) need an additional adhesive to interface with the skin and the measured ECG signals are vulnerable to skin deformations (FIG. 26). Integrated with a portable, custom-built signal processing system consisting of a 2-channel low-power analog front end and a built-in Bluetooth module, the adhesive patch can provide stable ECG monitoring after indoor workouts with obvious sweat secretion (FIG. 27). The heat rate (HR) of the human subject elevates from ~0.85 s to ~1.15 s after running and then recovers to ~0.9 s during rest after running (FIG. 27).

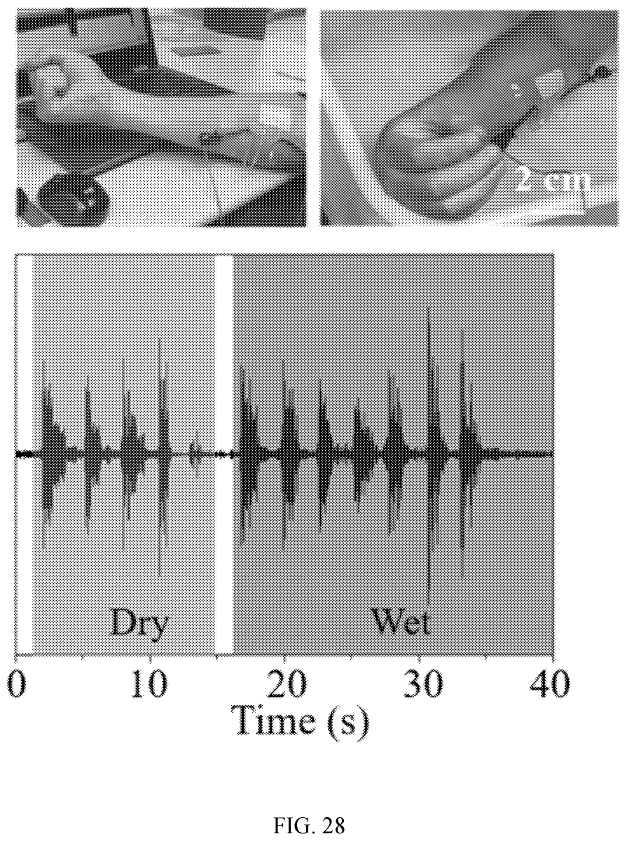

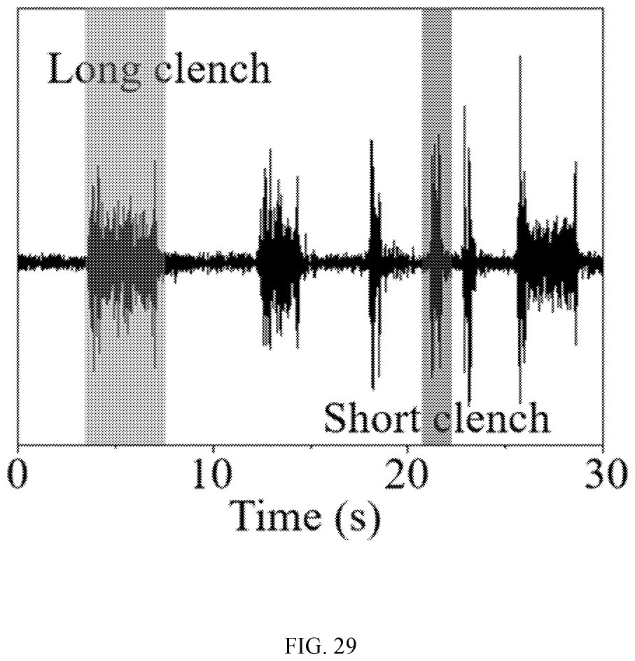

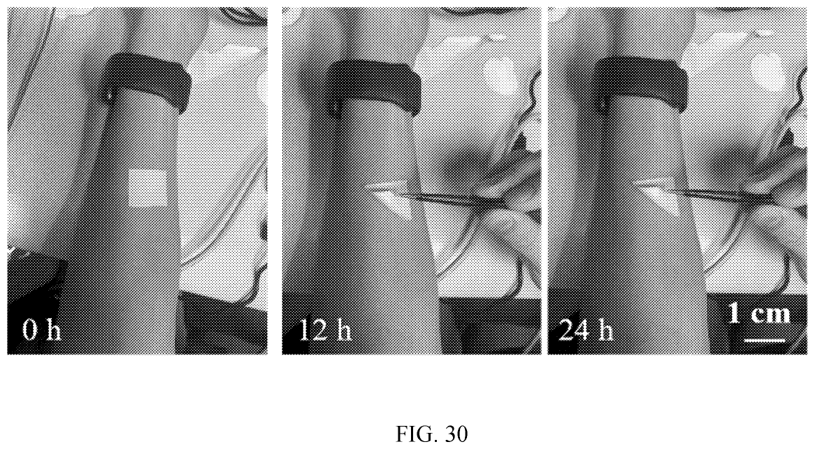

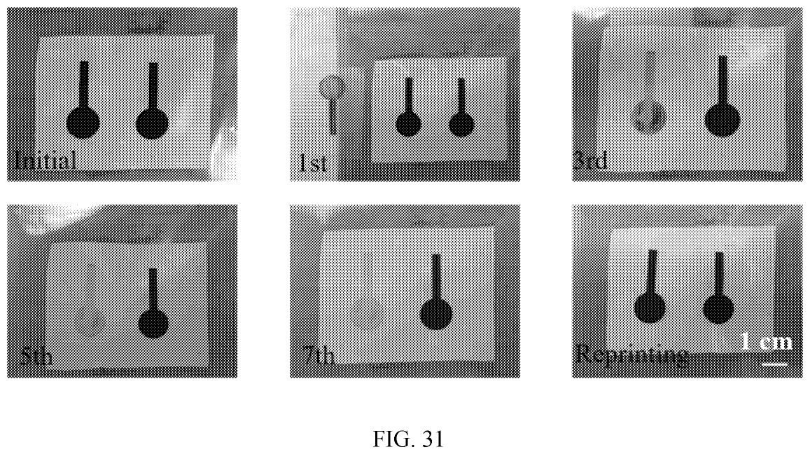

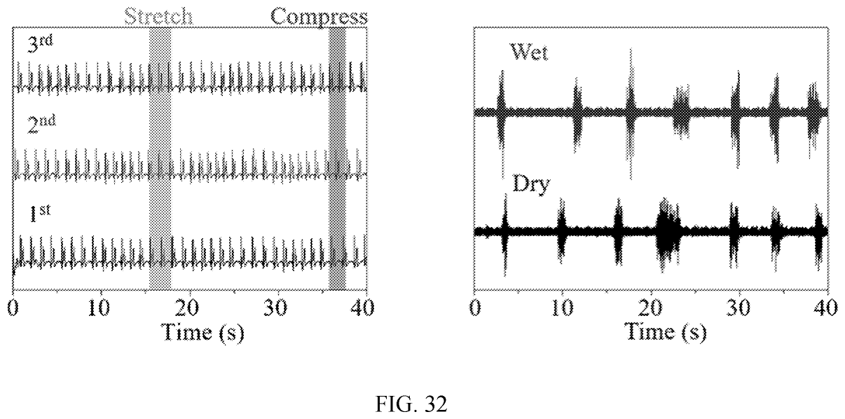

The hydrophobicity of PI/PDMS/PEIE also allows the composite to be used in wet or aqueous environments. Excellent sealing is achieved by a thin PDMS encapsulation and connecting the LIG electrode on the patch with thin silver stripes through a via filled with liquid metal that also avoids mechanical mismatch at the interface. EMG signals from clenching the first are captured with high fidelity (SNR of 21.8 or 22.3) in a dry state or submerged in water (FIG. 28). The short and long clench in the captured EMG signals can be accurately distinguished (FIG. 29). No signs of skin rashes are observed after 24 hours of wearing on the arm, indicating good biocompatibility and gas permeability of the patch (FIG. 30). The adhesive patch is also reprogrammable and reusable, which can be simply achieved by peeling off the LIG pattern with scotch tape followed by a laser rewriting process (FIG. 31). The patch is thinned down by ~20 μm after each laser scribing cycle and the one with an initial thickness of 100 m can be rewritten for ~3-4 times. The adhesive patch not only maintains a high SNR of 23.8 after three rewriting processes in the measured ECG but also exhibits more robust mechanical properties against deformation, which can be attributed to reduced thickness and mechanical stiffness (FIG. 32). Similarly, EMG signals can be captured by the rewritten patch in both dry and wet environments (FIG. 32).

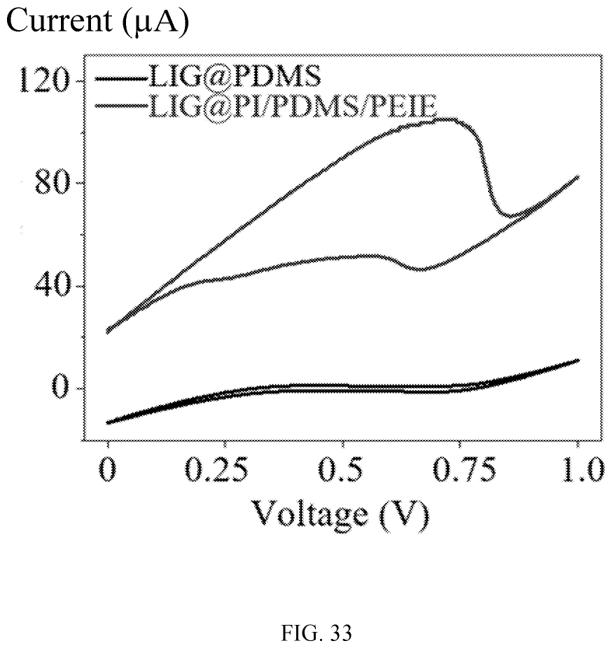

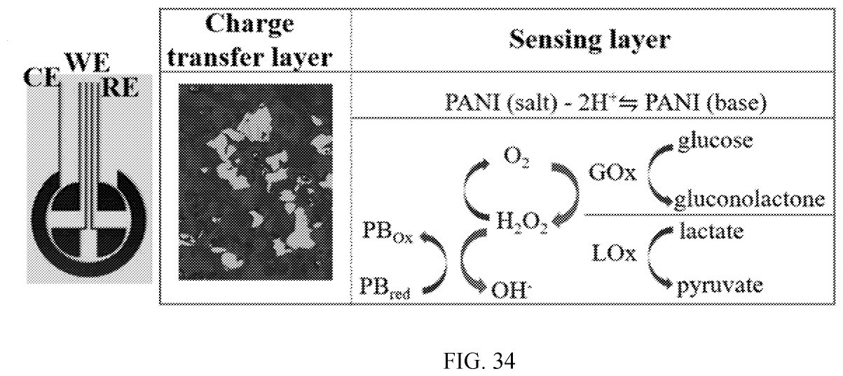

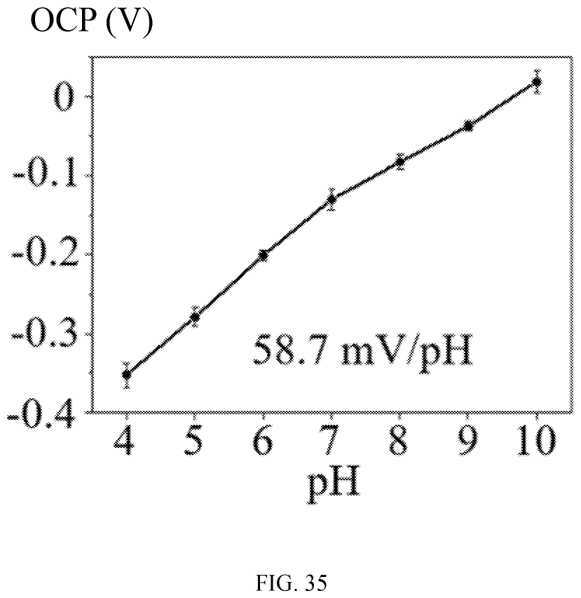

Electrochemical Sensing of pH, Glucose, and Lactate: Combined with simple functionalization (e.g., drop casting, electrodeposition, electroless plating, or adding additives in carbon precursors), the LIG with the porous structure and large specific surface area can be leveraged for energy storage and chemical/electrochemical biosensing. The importance of good hydrophilicity is revealed by depositing Prussian Blue (PB) on LIG@PI/PDMS/PEIE and LIG@PDMS with the cyclic voltammetry (CV) technique at a bias window from 0 to 1.0 V (FIG. 33). The hydrophilic LIG@PI/PDMS/PEIE with high porosity despite high resistance still exhibits a much higher current than the negligible value of LIG@PDMS, indicating the high efficiency of chemical modifications by electroplating. Compared with LIG@PDMS with reduced porosity and hydrophilicity, LIG@PI/PDMS/PEIE provides a multifunctional bioadhesive patch with excellent electrochemical sensing performance to detect various biomarkers such as pH, glucose, and lactic acid. The fabrication of the electrochemical sensors starts with electroplating Au nanoparticles (AuNP) on the LIG working electrode (WE) (FIG. 34) to improve the charge transfer efficiency, which is similar to spray-coating AgNWs on LIG as in the literature. The AuNP-modified LIG WE modified by electroplated PANI is then combined with an Ag/AgCl reference electrode in a two-electrode configuration to measure pH. Because the reaction potential of PANI depends on the proton concentration or the pH value, the measured open circuit potential (OCP) changes linearly with the pH value, with a sensitivity of 58.7 mV/pH (close to the Nernst limit) calculated from the slope of the linear fitting curve (FIG. 35). LIG@PI/PDMS/PEIE-based patch not only shows comparable pH sensing performance to that of the previously reported LIG@PI-based one but also owns intrinsic stretchability and adhesion for improved practicality of skin-interfaced electronics.

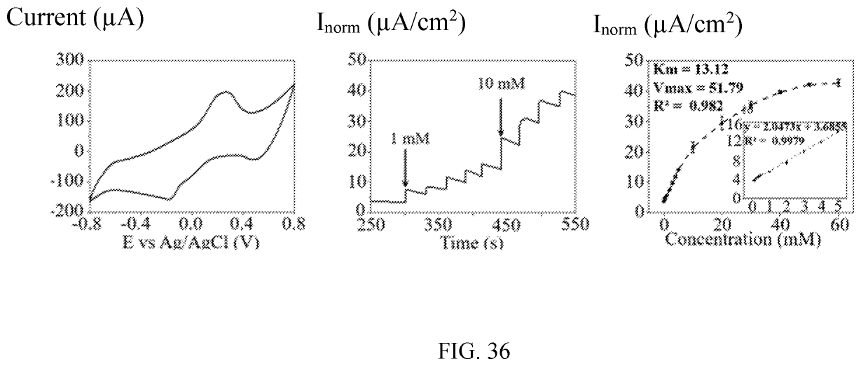

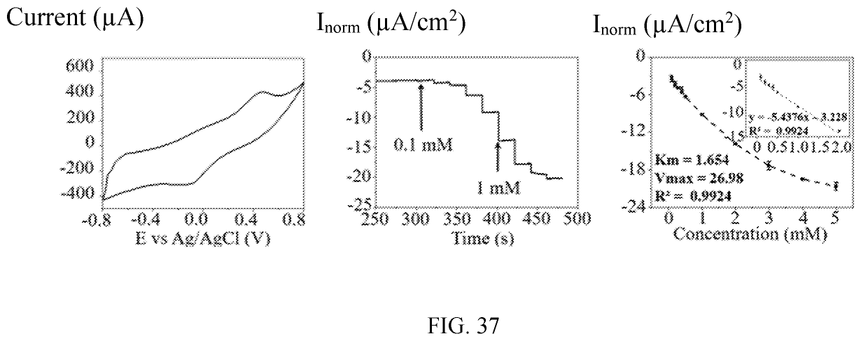

The immobilization of biorecognition elements or specific receptor molecules, such as enzymes or antibodies, on the LIG electrodes can also prepare electrochemical sensors for selective and sensitive detection of target analytes in biological samples. In a proof-of-the-concept demonstration to detect glucose and lactate, a thin layer of Prussian blue (PB) and gold nanoparticles (AuNPs) is first in situ co-electrodeposited on LIG using cyclic voltammetry to help immobilize enzymes due to its high surface area, stability, and biocompatibility. The favorable electrochemical properties (high electron conductivity and redox behavior) with efficient signal transduction and amplification can also increase selectivity and stability. The biomarker radicals or hydrogen peroxide produced after interacting biomarkers with the enzyme would oxidize or reduce Prussian blue to generate an electrical signal that can be measured and correlated to the biomarker concentration (FIG. 34). Further immobilizing the electrode with enzymes such as GOx and LOx in the WE provides the resulting electrochemical sensor in a three-electrode configuration to detect glucose (FIG. 36) and lactate (FIG. 37). The cyclic voltammetry (CV) tests of the glucose and lactate biosensor are measured between −0.8 and 0.8 V at a scan rate of 50 mV s⁻¹ in 0.2 M PBS (pH 6.5) solution with 1 M glucose and lactate, respectively (FIGS. 36 and 37). A bias potential of 0.26 V (vs. Ag/AgCl) for glucose and −0.1 V for lactate was employed to avoid interferences. The amperometric detection of consecutively injected glucose under stirring with the PB-GOx-modified LIG electrode at 0.26 V shows a linear dependence of the measured current ΔI (μA·cm²) on the glucose concentration from 100 μM to 5 mM, with a slope or sensitivity of 2.0473 μAcm⁻² mM⁻¹ and a correlation coefficient of 0.9927 (FIG. 36). Similarly, the PB-LOx-modified LIG electrode at −0.1 V detects the consecutively injected lactate from 100 μM to 2 mM with a sensitivity of 5.4376 μAcm⁻² mM⁻¹ and correlation coefficient of 0.9924 according to ΔI (pA·cm²)=−3.228-5.4376 [lactate](mM) (FIG. 37). The use of the Michaelis-Menten equation further reveals the kinetic behavior of the lactate and glucose sensor. This behavior analysis of the glucose and lactate oxidase enzymes allows the determination of kinetic parameters for further performance optimization of the enzymatic electrochemical sensors. The apparent Michaelis-Menten constant (Kₘ) and the maximum steady-state current response (Iₘₐₓ) are determined to be 13.12 mM and 51.79 pA·cm² (n=3) for glucose concentrations from 100 μM to 60 mM (or 1.654 mM and 26.98 pA·cm² for lactate from 100 μM to 5 mM). Those determined values for glucose and lactate sensors represent the great ability of the sensor to produce an electrical signal in response to a high concentration of glucose or lactate, with good sensitivity to detect lower glucose or lactate concentrations in the sweat. The linear working range and sensitivity of the LIG@PI/PDMS/PEIE-based electrochemical sensor are significantly larger than those based on LIG@PDMS, thanks to the well-preserved porosity for enzyme loading. A comparison of sensitivity, limit of detection (LOD), and working range to other transferred LIG-based (LIG@PDMS) electrochemical sensors (not limited to glucose) is given in Table 2.

TABLE 2
Comparison of the electrochemical sensor based on
PB/LIG@ PI/PDMS/PEI with previous work.

| | | | | Sensitivity | |
|---|---|---|---|---|---|
| Electrode Materials | Analyte | LOD | Range | (μAmM⁻¹cm⁻²) | Type |
| GOx/PtAuNP/LIG@PDMS | Glucose | 5 | 0-1.1 | 6.4 | Enzymatic |
| | | μM | mM | | |
| Pt-AuNPs/LIG@PDMS | Dopamine | 75 | 9.5 × 10⁻⁷- | 865.80 | Nonenzymatic |
| | | nM | 3 × 10⁻⁵ M | | |
| PDMS/LIG-Au-OPH | Methyl | 0.01 | 20-500 | N/A | Enzymatic |
| | parathion | μM | μM | | |
| OPH/AuNP/LIG@PDMS | Glucose | 26 | 10 μM- | 0.024 mA | Nonenzymatic |
| | | μM | 10 mM | mm⁻¹ | |
| GOx/PB/LIG@ | Glucose | | 100 μM | 2.0473 | Enzymatic |
| PI/PDMS/PEI | | | to 5 mM | | |

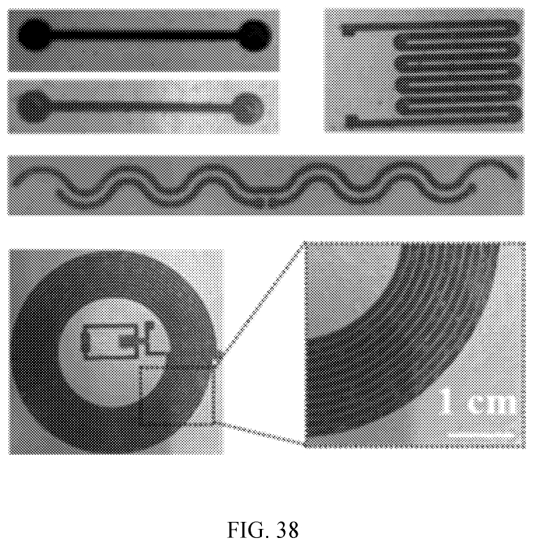

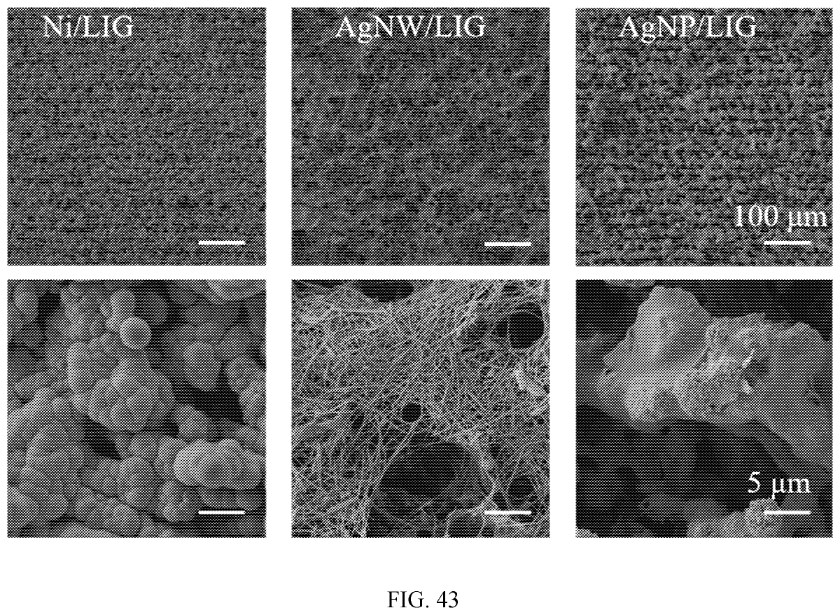

Enhanced Conductivity for DC Interconnects and RF Devices: Different from the resistive sensing unit in favor of higher resistance for precise measurement (e.g., strain/temperature sensing), the other functional components (e.g., wiring/interconnects, electrodes, heaters, or RF units) are supposed to have high conductivity. To obviate the need for radically different materials and fabrication processes, it is highly desirable to exploit and fabricate conductive LIG@PI/PDMS/PEIE patterns with spatial-dependent conductivity to avoid mechanical mismatch at the interface for improved durability and long-term use. Besides the ease of integration with circuit boards over “fly-over” wires, the minimized mechanical mismatch at the hetero-integrated interface could also avoid electrical failure under deformations. Due to the high hydrophilicity and porosity, the LIG in the composite can be selectively modified with highly conductive dopants, such as drop-casting of silver nanoparticles (AgNPs) ink, electrodeless plating of nickel, or air-spraying of silver nanowires (AgNWs) (FIG. 38). The sheet resistance of the modified LIG with nickel, AgNPs, and AgNWs reduces to 1.3, 3.1, 5.6Ω/□. The porosity of LIG decreases with the introduction of Ni or AgNPs, but was well maintained with the modification of AgNWs because of the larger dimension of AgNWs than the pore size (FIG. 43).

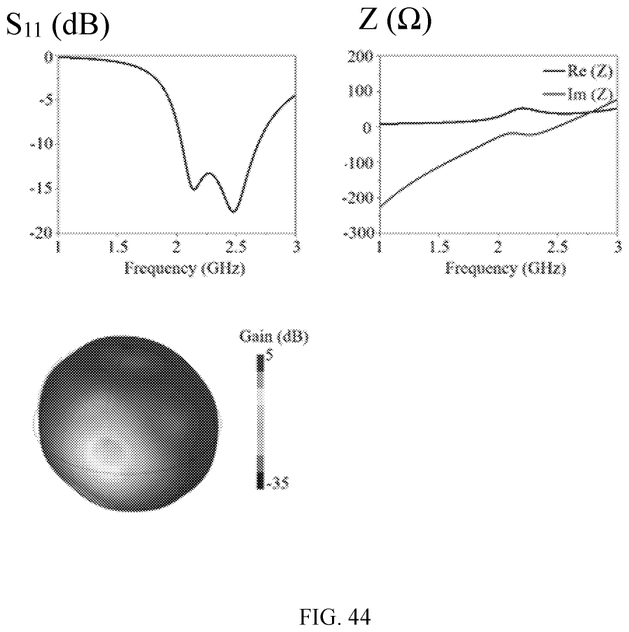

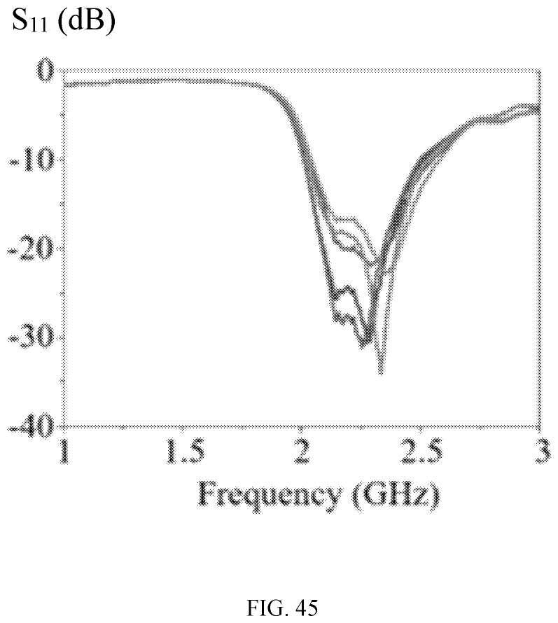

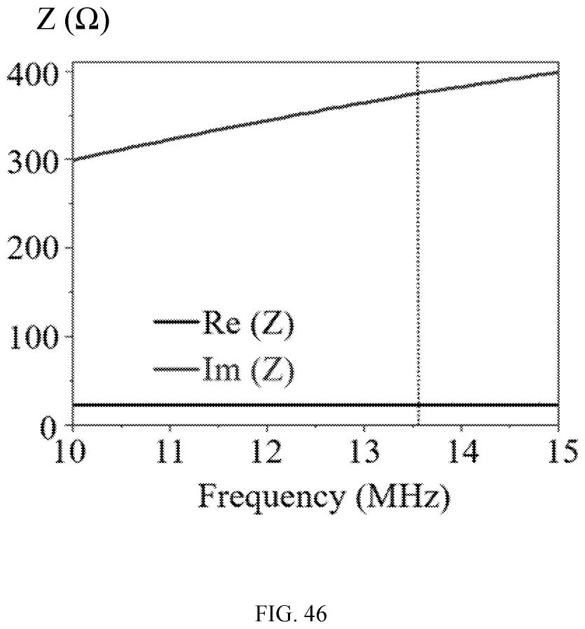

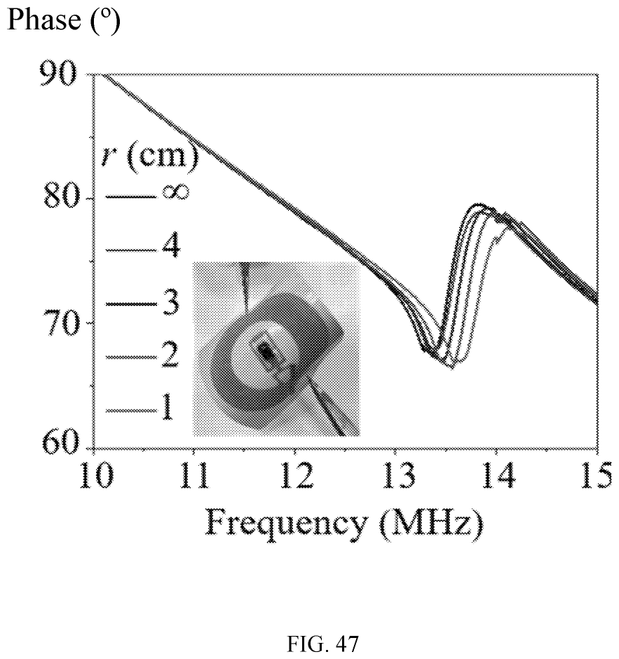

Electroless plating of Ni on LIG with winding structures results in a flexible heater that can rapidly heat up from 24° C. (i.e., the room temperature) to 105° C. as the applied bias voltage increases from 0 to 12 V. The relatively uniform temperature distribution in the heater confirms the uniform coating by electroless plating to improve the conductivity. RF devices are also in favor of high conductivity due to reduced ohmic loss. As a result, drop-casting AgNP ink on LIG followed by photonic sintering reduces the sheet resistance from 5241 to 3.1Ω/□, promising common RF devices such as wideband dipole antennas and near-field communication (NFC) coils. The flexible wideband dipole antenna based on AgNP/LIG shows characteristic “dual resonance” and a low reflection coefficient (S₁₁<−10 dB) at resonant frequencies (FIG. 36), whereas the one based on pristine LIG doesn't show observable resonance due to its large resistance. The dual resonance and low reflection of the AgNP/LIG-based dipole antenna contribute to a wide operational bandwidth (>0.4 GHz), which also remains unchanged upon bending deformations for potential applications in on-body wireless communication and RF energy harvesting. Simulations reveal the typical dipole-like impedance curves and radiation patterns (FIG. 44). Five antennas fabricated by the same procedure only exhibit a relatively small resistance variation of <10% and similar S₁₁ curves (FIG. 45), resulting in a variation of <3% for the radiation efficiency. An NFC coil with a designed inductance of ~5 nH can be combined in series with a commercial NFC chip with an internal capacitance of 27.5 pF to resonate at ~13.56 MHz for near-field communication with the smartphone (FIG. 36). The AgNP/LIG-based coil exhibits a modest Q factor of ~15.2 at 13.56 MHz (FIG. 46) with a broad frequency response for robust performance upon stretching. According to the min-phase method, the resonant frequency of the LC circuit can be determined by the inflection point of the reflection curve (FIG. 47). The negligible difference between the measured (13.56 MHz) and simulated (13.55 MHz) resonant frequency indicates excellent manufacturability of the approach. For a bending radius of 1 cm, the resonant frequency only slightly shifts from 13.55 MHz to 13.84 MHz, allowing for robust wireless communication and powering. As a result, laser scribing exhibits the advantages of high precision, low cost, and scalable production in antenna fabrication.

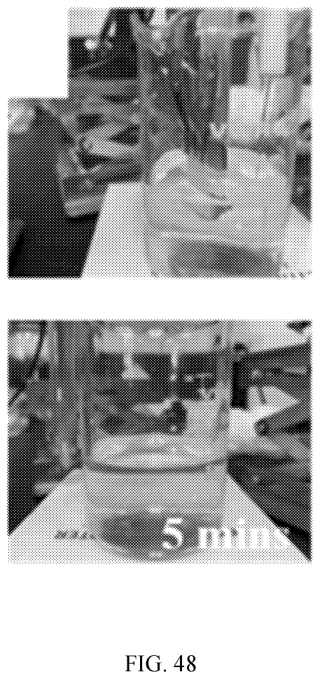

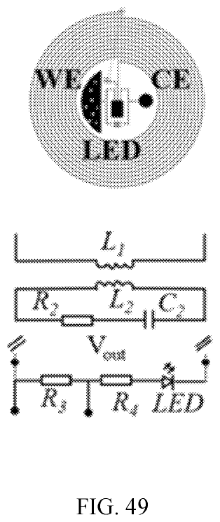

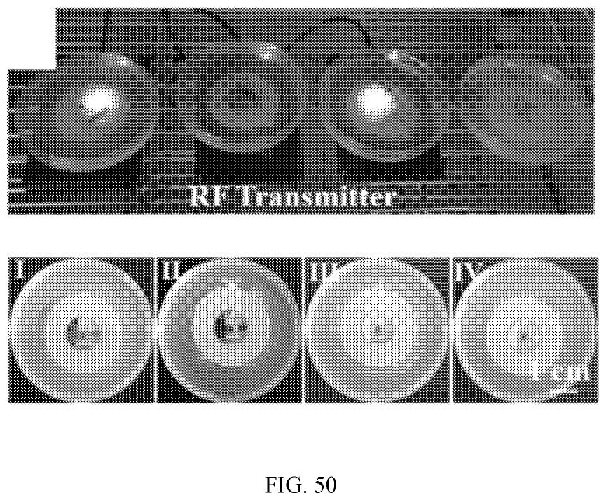

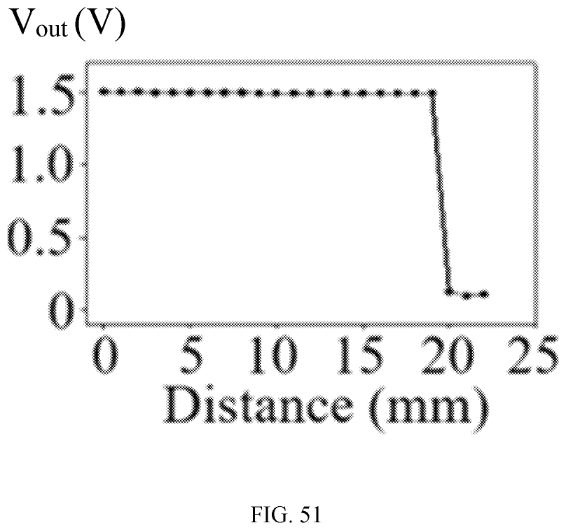

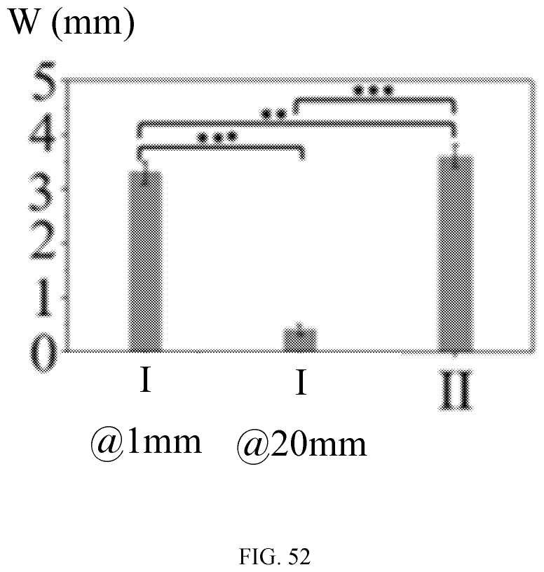

Multifunctional Skin-interfaced Platform Based on LIG@PI/PDMS/PEIE: Spatial-dependent modifications on the stretchable adhesive LIG@PI/PDMS/PEIE patch afford a wirelessly powered drug delivery system for wound healing. After immobilizing Cefazolin antibiotics on LIG electrodes with the assistance of polypyrrole (PPy) by electroplating, applying negative bias releases the negatively charged Cefazolin molecules, with PPy changed from the oxidized to reduced state. The efficacy of the controllable drug release is first evaluated by using fluorescein as a model drug molecule in preliminary experiments. Applying a positive bias of −1.5 V for 5 minutes changes the transparent electrolyte to green, indicating the effectiveness of electrically controlled fluorescein release (FIG. 48). The Cefazolin-modified LIG electrode is then combined with the aforementioned AgNP/LIG-based NFC coil for wirelessly controlled drug delivery (FIG. 49). Despite the modest Q factor, the coil ensures combined with the NFC chip can still generate an output voltage of 1.5 V after built-in rectification to power a UV LED and simultaneously trigger drug delivery. AgNP/LIG-based NFC devices include one with drug-loaded electrodes and LED (I), one with drug-loaded electrodes (II) or LED (III), and another blank one as the control (IV) (FIG. 50). Evaluation of the antibacterial effect of these four AgNP/LIG-based NFC devices relies on the zone of inhibition (ZOI). The harvested energy from an RF transmitter is applied between the work and counter electrodes to trigger drug delivery. In all cases, the wireless stimulation is set to 1 hour, and optical images of bacterial colonies and corresponding ZOI defined as the width of the bacteriostatic ring (w) are collected after 24 hours' culture in an incubator. Also, different working distances (i.e., 1 and 22 mm) between the device and transmitter for Group I are tested to investigate their influence on drug delivery. The output voltage of the coil after rectification and splitting remains ~1.5 V with an increasing distance up to 19 mm and then drops to ~0.2 V at 20 mm (FIG. 51). A zero ZOI in Groups III and IV indicates the inefficacy of bare electrical stimulation or LED illumination against S. aureus. In comparison, Group I shows a significantly larger ZOI, which is almost close to that of Group II (FIG. 52), indicating effective antibiotic delivery through the wirelessly controlled stimulation. However, increasing the working distance to 20 mm in Group I (i.e., I@20 mm) decreases the ZOI to ~0.3 mm due to reduced bias. Although UV LED doesn't directly inhibit S. aureus, it can serve as an indicator of the working distance to ensure effective drug delivery. Besides, it is possible to incorporate light-responsive hydrogels or photothermal effects to further boost the antibacterial performance. Considering the good adhesion, antibacterial capability, and possibly the electrical stimulation and photothermal effect, the AgNP/LIG-based adhesive device platform holds great potential in wound healing.

The system-level demonstration is showcased by integrating the adhesive LIG@PI/PDMS/PEIE patch with a miniaturized and portable electrochemical station on the forehead with a headband for real-time sweat glucose sensing (FIG. 48). The extended interconnects between the glucose sensor and the electrochemical station exploit LIG/AgNWs for reduced electrical resistance. The large difference in hydrophilicity between LIG and PI/PDMS/PEIE allows efficient sampling and transport of the sweat to the LIG-based glucose sensor, without the need for a microfluidic module. The sweat glucose concentration of a subject is continuously measured for 2 hours during indoor bicycling. The measured results show the elevated sweat glucose concentration (~0.145 mM) 1 hour later after having breakfast at 9:00 am (FIG. 49). With continuous workouts, the sweat glucose concentration slowly drops to 0.11 mM at 11:00 am due to the large consumption of calories. However, a slight increase in sweat glucose concentration is observed after that, possibly attributed to the glycogenolysis or gluconeogenesis process. The sweat glucose concentration is also compared with the blood glucose concentration measured with a commercial glucose sensor by finger pricking every 30 minutes between 9:30 and 11:30 am. The trend of increasing after breakfast and dropping during a workout, followed by a slight increase afterward in the blood glucose concentration, correlates well with the continuously measured sweat glucose concentration, with a delay of 30 mins. This delay is believed to arise from the diffusion process of glucose from blood to interstitial fluid and sweat. It may be possible to directly obtain an accurate blood glucose concentration by accounting for this time delay and other sweat parameters, such as pH and perspiration rate, to help calibrate the measurements.

### CONCLUSION

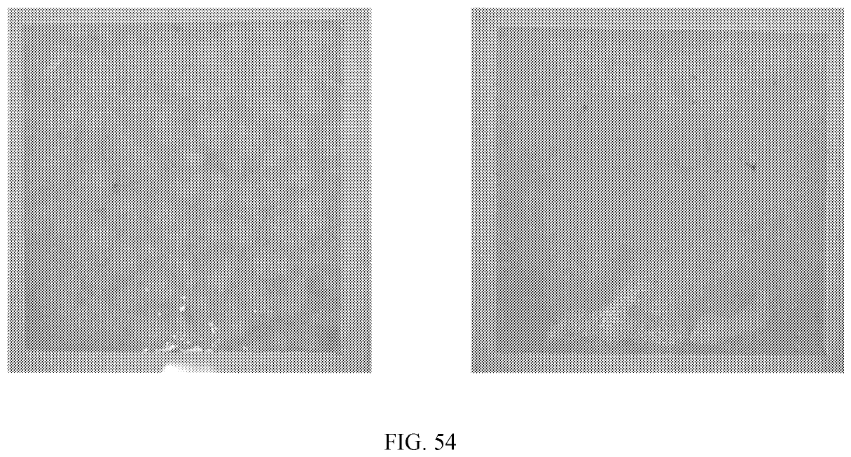

In this example, we developed a programmable, recyclable device platform based on PI/PDMS composites for multifunctional, skin-interfaced applications. The incorporation of PI and PEIE enabled laser processing of the composite for electrical or sensing functionalization and strong adhesion for direct skin-interfacing. LIG fabricated on the PI/PDMS composite allowed high-fidelity biopotential measurements of a subject during workouts or in a wet-skin state. Post-modifications of porous LIG on the adhesive composite with functional materials contributed to electrochemical sensing of bio-relevant parameters of sweat, including the pH value, glucose, and lactate concentration. Spatial-dependent modifications of LIG towards electrical and sensing functions were capable of realizing simple device-level integrations on the same material platform (i.e., LIG@PI/PDMS/PEIE). Successful real-time monitoring of ECG, EMG, and sweat glucose concentration based on the LIG@PI/PDMS/PEIE-based sensing platform has been demonstrated. This material design strategy may also be applicable to other polymers such as, but not limited to, thermoplastic polyurethane (TPU) or styrene-ethylene-butylene-styrene (SEBS), resulting in functional composites with different mechanical and electrical properties for broad application scenarios (FIG. 54).

It should be understood that modifications to the embodiments disclosed herein can be made to meet a particular set of design criteria. For instance, the number of or configuration of components or parameters may be used to meet a particular objective.

It will be apparent to those skilled in the art that numerous modifications and variations of the described examples and embodiments are possible in light of the above teachings of the disclosure. The disclosed examples and embodiments are presented for purposes of illustration only. Other alternative embodiments may include some or all of the features of the various embodiments disclosed herein. For instance, it is contemplated that a particular feature described, either individually or as part of an embodiment, can be combined with other individually described features, or parts of other embodiments. The elements and acts of the various embodiments described herein can therefore be combined to provide further embodiments.

It is the intent to cover all such modifications and alternative embodiments as may come within the true scope of this invention, which is to be given the full breadth thereof. Additionally, the disclosure of a range of values is a disclosure of every numerical value within that range, including the endpoints. Thus, while certain exemplary embodiments of the device and methods of making and using the same have been discussed and illustrated herein, it is to be distinctly understood that the invention is not limited thereto but may be otherwise variously embodied and practiced within the scope of the following claims.

What is claimed is:

1. A method of fabricating a device, the method comprising:
forming a mixture comprising:
one or more polymer precursors capable of forming laser-induced graphene (LIG),
one or more elastomeric prepolymers, and
one or more adhesion-promoting additives;
curing the mixture to form a polymer composite; and
laser scribing the polymer composite to pattern an integrated LIG network.

2. The method of claim 1, wherein the polymer precursors are selected from the group consisting of polyimide, polyamide-imide, polyether sulfone, polyphenylene sulfide, polyethylene terephthalate, polyethylene naphthalate, and combinations thereof.

3. The method of claim 1, wherein the polymer precursors include polyimide.

4. The method of claim 1, wherein the elastomeric prepolymers are selected from the group consisting of polydimethylsiloxane, thermoplastic polyurethane, styrene-ethylene-butylene-styrene, and combinations thereof.

5. The method of claim 1, wherein the elastomeric prepolymers include polydimethylsiloxane.

6. The method of claim 1, wherein the adhesion-promoting additives are selected from the group consisting of polyethylenimine ethoxylated, polyethyleneimine, polydopamine, tannic acid, and combinations thereof.

7. The method of claim 1, wherein the adhesion-promoting additives include polyethylenimine ethoxylated.

8. The method of claim 1, wherein the polymer precursors include polyimide, the elastomeric prepolymers include polydimethylsiloxane, and the adhesion-promoting additives include polyethylenimine ethoxylated.

9. The method of claim 1, wherein the mixture comprises from 50 wt % to 75 wt % of the polymer precursors and from 25 wt % to 50 wt % of the elastomeric prepolymers, relative to the total combined weight of the polymer precursors and elastomeric prepolymers.

10. The method of claim 1, wherein the mixture comprises 25 μL to 30 μL of the adhesion-promoting additives per 10 g of the elastomeric prepolymers.

11. The method of claim 1, further comprising:
functionalizing the LIG network with conductive nanomaterials.

12. The method of claim 8, wherein the conductive nanomaterials are selected from the group consisting of carbon nanotubes, graphene oxide, and reduced graphene oxide.

13. The method of claim 8, wherein the conductive nanomaterials are metal nanoparticles selected from the group consisting of gold, silver, platinum, palladium, manganese, zinc, copper, nickel, cobalt, iron, titanium, molybdenum, tungsten, magnesium, aluminum, lead, and combinations thereof.

14. The method of claim 1, further comprising:
functionalizing the LIG network with enzymes.

15. The method of claim 11, wherein the enzymes are selected from the group consisting of glucose oxidase and lactate oxidase.

16. The method of claim 1, further comprising:
peeling off and removing an uppermost portion of the polymer composite with the integrated LIG network; and
laser scribing an underlying portion of the polymer composite to pattern a second integrated LIG network.

17. A device fabricated from the method of claim 1.

18. The device of claim 17, wherein the device exhibits intrinsic adhesion and stretchability.
